(19) United States
(12) Patent Application Publication
(10) Pub. No.: US 2026/0041787 A1
(43) Pub. Date: Feb. 12, 2026

(54) GENE THERAPY FOR LEVODOPA-INDUCED DYSKINESIA

(71) Applicant: Northwestern University, Evanston, IL (US)

(72) Inventors: Dalton James SURMEIER, Evanston, IL (US); Weixing Shen, Evanston, IL (US); Shenyu Zhai, Evanston, IL (US); Tatiana Tkatch, Evanston, IL (US); Qiaoling Cui, Evanston, IL (US); Zhong Xie, Evanston, IL (US)

(21) Appl. No.: 19/230,790

(22) Filed: Jun. 6, 2025

Related U.S. Application Data
(60) Provisional application No. 63/657,435, filed on Jun. 7, 2024.

(51) Int. Cl. A61K 48/00; A61P 25/14; C12N 15/113; C12N 15/86

(52) U.S. Cl. CPC A61K 48/005; A61P 25/14; C12N 15/113; C12N 15/86; C12N 2310/531; C12N 2750/14143

(57) ABSTRACT

The present disclosure provides compositions and methods for the treatment of levodopa-induced dyskinesia (LID) therewith. In particular, the present disclosure provides methods for treating LID by knocking down expression of the gene (GRIN2B) coding for the GluN2B subunit of N-methyl-d-aspartate receptors in indirect pathway spiny projection neurons (iSPNs) to attenuate the development and expression of LID without compromising the treatment of levodopa.

### CROSS-REFERENCE TO RELATED APPLICATIONS

This application claims the benefit of U.S. Provisional Patent Application No. 63/657,435, filed Jun. 7, 2024; which is incorporated by reference herein in its entirety.

### STATEMENT REGARDING FEDERALLY SPONSORED RESEARCH OR DEVELOPMENT

This invention was made with government support under grant number NS034696 awarded by the National Institutes of Health. The government has certain rights in the invention.

### SEQUENCE LISTING PARAGRAPH

The text of the computer readable sequence listing filed herewith, titled “NWEST_43209_202_SequenceListing.xml”, created Oct. 7, 2025, having a file size of 5,149 bytes, is hereby incorporated by reference in its entirety.

### FIELD

The present disclosure provides compositions and methods for the treatment of levodopa-induced dyskinesia (LID) therewith. In particular, the present disclosure provides methods for treating LID by knocking down expression of the gene (GRIN2B) coding for the GluN2B subunit of N-methyl-d-aspartate receptors in indirect pathway spiny projection neurons (iSPNs) to attenuate the development and expression of LID without compromising the treatment of levodopa.

### BACKGROUND

Parkinson's disease (PD) is the second most common neurodegenerative disorder. In the United States alone, more than one million Americans are living with PD and this number is projected to increase to 1.2 million by 2030 due to the aging population. PD is characterized as a progressive neurological disorder that primarily affects movement, causing tremors, stiffness, and difficulty with balance and coordination.

One prominent aspect of PD is the dual motor and non-motor symptomology that arises from the loss of dopamine-producing neurons in the substantia nigra region of the brain. As a result, non-motor symptoms include cognitive changes such as memory difficulties, slowed thinking, and executive dysfunction, with some patients developing PD dementia. In terms of motor symptoms, movement is significantly affected in PD due to the degeneration of dopamine-producing neurons which are critical for motor control. This is because dopamine is the key neurotransmitter in the facilitation and communication between the substantia nigra and the striatum, which are essential regions within the basal ganglia for coordinating smooth and controlled muscle movements. Therefore, the progressive loss of these dopamine-producing neurons leads to a deficiency of dopamine, disrupting the normal signaling pathways within the basal ganglia. This disruption results in the hallmark motor symptoms of PD, including tremors, bradykinesia (slowness of movement), rigidity (muscle stiffness), and postural instability (balance problems). Without adequate dopamine, the basal ganglia cannot properly regulate motor functions, leading to the characteristic difficulties in initiating and controlling movements.

In the early stages of PD, when a substantial population of dopaminergic neurons remain, boosting their release of dopamine (DA) by administration of its blood-brain barrier penetrant precursor, levodopa (L-3,4-dihydroxyphenylalanine), effectively alleviates motor symptoms. However, as the disease progresses, higher oral doses of levodopa are required to achieve symptomatic benefit and as a result, brain concentrations of DA become dysregulated—rising to abnormally high levels for hours and then falling back to very low levels as systemic levodopa wains. As the disease progresses and dopaminergic neurons continue to decline, patients often experience fluctuations in their response to levodopa, leading to motor complications. One such complication is levodopa-induced dyskinesia (LID), which are involuntary, erratic, and often writhing movements that can occur as a complication of long-term levodopa use. Dyskinesias typically emerge during the later stages of the disease, reflecting the cumulative impact of prolonged dopaminergic therapy and disease progression. Management of LID may involve adjusting the dosage and timing of levodopa, adding other medications to smooth out the response, or considering advanced therapies such as deep brain stimulation (DBS) which involves invasive surgery. Nevertheless, adjusting levodopa or introducing new medications to a PD patient can cause a host of issues such as, fluctuations and worsening of motor control, increased side effects, and the potential for new complications. Likewise, the effectiveness of DBS can vary significantly among patients, and this variability makes it difficult to predict outcomes for individual patients. In some instances, DBS may even exacerbate these non-motor issues and the surgical risks include potential complications such as infection, bleeding, and stroke, which, although rare, can be serious. In the long term, DBS does not stop the progression of Parkinson's disease, and patients may develop new symptoms that are unresponsive to the treatment.

Therefore, there is a critical need for treatments that effectively address levodopa-induced dyskinesia (LID) in Parkinson's disease without introducing additional deficits or interfering with the efficacy of levodopa. Such treatments would enhance motor control and quality of life for patients by managing dyskinesias while preserving the benefits of existing dopaminergic therapies. This would ensure that patients maintain optimal symptom control with minimal side effects, facilitating better long-term management of the disease.

### SUMMARY

Provided herein are compositions and methods for the treatment of levodopa-induced dyskinesia (LID) therewith. In particular, the present disclosure provides methods for treating LID by knocking down expression of the gene (GRIN2B) coding for the GluN2B subunit of N-methyl-d-aspartate receptors in indirect pathway spiny projection neurons (iSPNs) to attenuate the development and expression of LID without compromising the treatment of levodopa.

Embodiments of the present disclosure include a method for treating Levodopa-induced dyskinesia (LID), in a subject in need thereof, the method comprising: administering a GRIN2B inhibitor to the subject.

In some embodiments, the GRIN2B inhibitor is administered into an indirect pathway spiny projection neuron (iSPN).

In some embodiments, the GRIN2B inhibitor reduces GluN2B protein levels.

In some embodiments, the GRIN2B inhibitor is an inhibitor of GRIN2B expression.

In some embodiments, the GRIN2B inhibitor is a nucleic acid inhibitor of GRIN2B expression.

In some embodiments, the GRIN2B inhibitor knocks down the expression of GluN2B protein to suppress the induction of iSPN adenosine 2a receptors (A2aRs)-dependent long-term potentiation (LTP).

In some embodiments, the nucleic acid inhibitor is a microRNA (miRNA), a small interfering (siRNA), a short hairpin RNA (shRNA), an anti-sense RNA (asRNA), a competing endogenous RNA (ceRNA), a long non-coding RNA (lncRNA) and a ribozyme. In some embodiments, the nucleic acid inhibitor is dispersed into a vector. In other embodiments, the vector is a viral vector. In further embodiments, the viral vector is an adeno-associated viral vector (AAV), a adenoviral vector, a lentiviral vector, a non-viral vector, a human-compatible vector, a herpes simplex virus vector and a retroviral vector.

In some embodiments, the subject is afflicted with the neurodegenerative disease associated with an upregulation of GluN2B-containing N-methyl-D-aspartate receptors (NMDARs).

In some embodiments, the neurodegenerative disease from Parkinson's disease.

In some embodiments, the subject is a human. In some embodiments, the subject was administered or is currently being a therapeutic agent.

In some embodiments, the therapeutic agent administered is levodopa (L-3,4-dihydroxyphenylalanine). In other embodiments, administering levodopa to the subject induces fluctuations of dopamine (DA) levels in a subject; wherein large and uncontrolled fluctuations in DA levels results in levodopa-induced dyskinesia (LID).

In further embodiments, the administering levodopa to the subject increases the expression of the GluN2B-containing NMDARs in iSPNs. In some embodiments, the increased expression of the GluN2B-containing NMDARs in iSPNs during fluctuations of DA levels results in the upregulation of glutamatergic synapses and the induction of A2aR-dependent LTP.

In some embodiments, the GRIN2B inhibitor knocks down the expression of GluN2B to suppress the induction of iSPN adenosine 2a receptors (A2aRs)-dependent long-term potentiation (LTP) to diminish the induction and expression of levodopa-induced dyskinesia (LID).

In some embodiments, the GRIN2B inhibitor is an Intraperitoneal (IP) injection.

Provided herein are compositions and methods treating PD with levodopa and an GRIN2B inhibitor.

In some embodiments, said GRIN2B inhibitor is administered repeatedly to the subject. In other embodiments, said GRIN2B inhibitor is administered once to the subject.

In some embodiments, wherein said GRIN2B inhibitor is administered during the low levels of DA to the subject after levodopa is administered.

In some embodiments, the use of GRIN2B inhibitor for use in the manufacture of a medicament for the treatment of a neurodegenerative disease in a subject in need thereof.

In some embodiments, the use of GRIN2B inhibitor for treatment of a neurodegenerative disease in a subject in need thereof.

In some embodiments, the use of GRIN2B inhibitor for use in the manufacture of a medicament for the treatment of Levodopa-induced dyskinesia (LID) in a subject in need thereof.

In some embodiments, the use of GRIN2B inhibitor for use in the treatment of Levodopa-induced dyskinesia (LID) in a subject in need thereof.

### BRIEF DESCRIPTION OF THE DRAWINGS

FIGS. 1A-J. Upregulation of iSPN GluN2B-containing NMDARs after LID induction. (A) A light microscopic image of a coronal section illustrating the loss of immunoreactivity for tyrosine hydroxylase (TH, red) after the unilateral medial forebrain bundle (MFB) 6-OHDA lesioning. (B) Experimental timeline indicating the initial monition of 6-OHDA-lesioned mice with robust lesions [>85% striatal tyrosine hydroxylase (TH) loss; after 21-28 days with control recording (no levodopa)] were given either short (3-5 days) or long (12-14 day) regimen of levodopa at a dose inducing dyskinesia in all animals (3 mg/kg/day, i.p.), sacrificed and recorded. (C) Graphs indicating the averaged EPSC traces recorded at −70 mV (AMPAR-EPSC) and then +40 mV in the presence of the AMPAR antagonist NBQX (10 μM, NMDAR-EPSC) from a representative naïve, lesioned or dyskinetic iSPN. EPSC traces were normalized to the same height of NMDAR-EPSCs. The decay of these NMDAR currents were typically biexponential, as shown in the semi-logarithm plot (lower right). The NMDAR current in the levodopa-treated iSPN decayed significantly slower than untreated iSPNs. (D) A box plot summary of the ratio of the NMDAR EPSC (measured at the holding potential of +40 mV in the presence of 10 μM NBQX) to the AMPAR EPSC (measured at −70 mV) in iSPNs from naïve (n=7, from 4 mice), lesioned (n=6, from 4 mice) and dyskinetic (n=5, from 4 mice) states. The box plot boxes indicate upper and lower quartiles; whiskers specify upper and lower 90%. *p<0.05, Mann-Whitney rank sum test. (E) A box plot summary of the weighted iSPN NMDAR EPSC decay time constants from naïve, lesioned and dyskinetic states. *p<0.05, Mann-Whitney test. (F) A bath application of GluN2A selective antagonist Zn2+ (100 μM with 10 mM tricine) decreased the size of the NMDAR currents and prolonged its deactivation. (G) A bath application of the GluN2B selective antagonist Ro 25-6981 (1 μM) indicating a decrease in the size of the NMDAR currents but did not change their deactivation kinetics. (H) A box plot summary of alterations of iSPN NMDAR current decay kinetics by the selective GluN2A antagonist (n=5, from 3 mice, p<0.05, Wilcoxon signed rank test) and the selective GluN2B antagonist (n=6, from 4 mice, p>0.05, Wilcoxon test). *p<0.05; ns not significant. (I) NMDAR EPSCs from a naïve iSPN in control (black trace) and in 25 μM MK-801 (red trace) showing the reduction of the peak amplitude and the acceleration of the de-activation and the charge transfer (middle). In MK-801 the ratio of charge at the time of the EPSC peak (a) to the total charge (b) indicates the estimate of the probability of channels having opened by the time of the peak. (J) A box plot indicating the peak open probabilities (Po) of GluN2A-type NMDARs (0.52, N2A, blue dashed line), GluN2B-type NMDARs (0.24, N2B, red dashed line) and GluN2A/B-type NMDARs (0.28, N2A/B, black dashed line). The peak open probabilities of naïve (n=5, from 3 mice) and dyskinetic (n=6, from 3 mice) iSPNs fell near the value GluN1/N2A/B or GluN1/N2B receptors.

FIGS. 2A-F. GluN2B-preferring inhibitor suppresses more NMDAR currents in levodopa-treated iSPNs than untreated iSPNs. (A-B) NMDAR currents were measured at the holding potential of −40 mV in the presence of 10 μM AMPAR antagonist NBQX and 10 μM GABAA antagonist gabazine. In dSPNs, bath application of GluN2B selective inhibitor Ro 25-6891 (1 μM) reduced NMDAR currents by a similar size from naïve, lesioned and dyskinetic mice. (C) A box plot summary of the percent modulation of dSPN NMDAR currents from naïve (n=5, from 4 mice), lesioned (n=5, from 3 mice) and dyskinetic (n=6, from 4 mice). p>0.05, Mann-Whitney test. ns not significant. (D-E) In iSPNs, Ro 25-6891 had more percent reduction in NMDAR currents from dyskinetic mice than naïve and lesioned mice. (F) A box plot summary of the percent modulation of iSPN NMDAR currents from naïve (n=6, from 4 mice), lesioned (n=5, from 4 mice), dyskinetic short-duration (3-5 days) (n=7, from 5 mice) and dyskinetic long-duration (12-14 days) (n=5, from 4 mice). * p<0.05, Wilcoxon test.

FIGS. 3A-H. Enrichment of GluN2B-containing NMDARs creates AMPA-deficient silent synapses on iSPNs. Representative traces showing 40 consecutive EPSCs at +40 mV and −70 mV from the levodopa-treated dSPN (a) and iSPN (d). The EPSCs were elicited by minimal electrical stimulation measured at −70 mV (AMPAR-EPSC) and +40 mV (NMDAR EPSC) in the presence of the AMPAR antagonist NBQX (10 μM). Plots of AMPAR and NMDAR EPSC amplitudes in the example dSPN (b) and iSPN (e). (c) Box plot sum of dSPN silent synapse percentage from lesioned (n=4, from 3 mice) and dyskinetic states (n=4, from 3 mice). The percent of silent synapses was estimated using the equation: 1−[ln(F−70)/ln(F+40)], where F−70 is the failure rate at −70 mV and F+40 is the failure rate at +40 mV. ns not significant, Wilcoxon test. (f) Box plot summary of the percentage of iSPN silent synapses measured from lesioned (n=6, from 5 mice), dyskinetic on-state (n=5, from 5 mice), dyskinetic off-state short-duration treated (3-5 days, n=5, from 4 mice) and dyskinetic offstate long-duration treated (12-14 days, n=6, from 5 mice) animals. *p<0.05, **p<0.01, Wilcoxon test. (g) Plots of iSPN AMPAR (at −70 mV) and NMDAR (at +40 mV) EPSCs from naive, lesioned, dyskinetic (short duration) and dyskinetic (long-duration) conditions. (h) Box plot summary of the ratio of CVNMDAR to CVAMPAR from naive (n=7, from 5 mice), lesioned (n=6, from 5 mice), dyskinetic short (n=6, from 5 mice) and dyskinetic long (n=5, from 5 mice) states. *p<0.05, Wilcoxon test.

FIGS. 4A-F. Up-regulation of iSPN GluN2B-containing NMDARs promotes LTP. (a) The experimental configuration. (b) Schematic illustrating the LTP induction protocol. (c) LTP was induced by a pre-post timing pairing in iSPNs. Plots show EPSP amplitude and input resistance (Ri) as a function of time. Scale bars: 2 mV×50 ms. (d) In the lesioned state, a pre-post timing pairing did not lead to LTP induction, whereas the same protocol revealed LTP in the dyskinetic state. Data are represented as mean±SEM. Plot of the average EPSP amplitudes as a function of time. Lesioned n=6, from 6 mice; dyskinetic n=6, from 6 mice, p<0.05, Mann-Whitney rank sum test. (e) In dyskinetic state, pre-post pairing led to LTP; the LTP was disrupted by addition of the selective A2aR antagonist tozadenant (1 μM). Data are represented as mean±SEM. Plot of the average EPSP amplitudes as a function of time. Dyskinetic n=6, from 6 mice; tozadenant n=5, from 5 mice, p<0.05, Mann-Whitney test. (f) In naive state, selective GluN2B antagonist Ro 25-6891 (1 μM) did not disrupt LTP induction. However, the induction was blunted by the same inhibitor in the dyskinetic state. Data are represented as mean±SEM. Plot of the average EPSP amplitudes as a function of time. Naive n=5, from 5 mice; dyskinetic n=6, from 6 mice, p<0.05, Mann-Whitney test.

FIGS. 5A-M. Antagonism of A2aR and GluN2B NMDARs attenuates the induction of dyskinetic behaviors. (a) Experimental timeline. Note that the treatment of tozadenant started in the off-state. (b) Plot of sum of axial, limb and orolingual AIM scores (levodopa+vehicle n=8; levodopa+tozadenant n=8, mean±SEM) as a function of time. Systemic treatment with tozadenant (30 mg kg-1, i.p.) produced a significant overall reduction in AIM scores (time p=0.0158, F(7, 98)=2.62; group p=0.114, F(1, 14)=2.83; interaction p<0.0001, F(7, 98)=5.5; Fisher p<0.05 on Day 11, 14 and 15. Repeated measure two-way ANOVA and post hoc Fisher). (c) Plot of sum of mouse AIM scores (n=8, mean±SEM) as a function of time on Day 15 (time p<0.0001, F(5, 70)=30.8; group p=0.044, F(1, 14)=4.88; interaction p=0.0109, F(5, 70)=1.8; Fisher p<0.05 at 60 and 80 min. Repeated measure two-way ANOVA followed by post hoc Fisher). (d) Representative confocal image showing the expression of AAV-DIO-GRIN2B shRNA/GFP in the DLS of an Adora2-Cre mouse. Scale bar=1 mm. (e, left) Confocal image showing GluN2B immunoreactivity (red) in the contralateral striatum that did not have GRIN2B shRNA/GFP expression. Scale bar=20 μm. (e, right) Confocal image showing GRIN2B shRNA/GFP (green) was expressed in ˜40% of total cells in the injected striatal region, consistent with Cre-dependent cell type specific expression in Adora2-Cre mice. Scale bar=20 μm. (f) High-magnification merged image of GluN2B immunoreactivity (red) and GRIN2B shRNA/GFP (green). Note that the GluN2B immunoreactivity, which was concentrated in the dendrites, did not overlap at all with GRIN2B shRNA/GFP-positive dendrites. Scale bars=20 μm. Knocking down GluN2B in infected iSPNs occluded selective antagonist blockade (g) and accelerated NMDAR current decaying (h). NMDAR currents were measured at the holding potential of −40 mV in the presence of 10 μM AMPAR antagonist NBQX and 10 μM GABAA antagonist gabazine. (g) Box plot summary. Scrambled control n=5, from 3 mice; GRIN2B shRNA n=7, from 5 mice; p<0.01, Mann-Whitney test. (h) Box plot summary. Scrambled control n=5, from 3 mice; GRIN2B shRNA n=7, from 5 mice; p<0.01, Mann-Whitney test. (i) Experimental timeline. (j and k) Plots of sum of AIM scores (levodopa+scrambled shRNA (control) n=6; levodopa+GRIN2B shRNA n=6; mean±SEM) as a function of days (j) or hours (k). (j) Time p<0.0001, F(7, 70)=6.33; group p=0.0021, F(1, 10)=16.90; interaction p<0.0001, F(7, 70)=7.60; repeated measure two-way ANOVA and post hoc Bonferroni's test. (k) Time p<0.0001, F(5, 50)=22.47; group p=0.001, F(1, 10)=20.82; interaction p<0.0001, F(5, 50)=8.39; repeated measure two-way ANOVA followed by Bonferroni's post test. (1) Experimental timeline. (m) Plot of sum of AIM scores (levodopa+scrambled shRNA (control) n=6; levodopa+GRIN2B shRNA n=6; mean±SEM) as a function of time. (time p=0.0009, F(4, 40)=5.82; group p=0.0045, F(1, 10)=13.31; interaction p=0.0005, F(4, 40)=6.31; repeated measure two-way ANOVA followed by Bonferroni's post test.).

FIG. 6. Prolonged treatment (12-14 days) of levodopa promotes induction of iSPN LTP in the LID off-state. LTP was induced with the pre-post pairing protocol in iSPNs from levodopa treated lesioned animals (n=5; p<0.05, Wilcoxon test). Plot of normalized EPSP amplitude as a function of time. Data are shown as mean±SEM. The dashed line represents the average of EPSP amplitude before induction. The vertical bar indicates STDP induction.

FIG. 7 Co-treatment of the A2aR antagonist tozadenant does not jeopardize the anti-parkinsonian effect of levodopa. The reduction in AIM scores had no effect on the anti-parkinsonian benefit of levodopa, as forelimb use asymmetry was improved by levodopa only or when co-treated with tozadenant (levodopa only n=8 mice, p<0.01, Wilcoxon test; levodopa+tozadenant n=8 mice, p<0.01, Wilcoxon test). **p<0.01.

FIGS. 8. iSPN intrinsic properties were not altered with GluN2B knockdown in LID off-state. (a) representative current clamp recording traces showing the change of membrane potentials and evoked action potentials in response to −120 pA, 220 pA and 400 pA current injection steps. Left, scrambled control (black); right, GRIN2B shRNA (red). (b) The firing induced by a series of 0-400 pA current injection steps was not changed with GRIN2B shRNA (top middle; n=8 cells/3 mice for each group). There is no difference in (c) resting membrane potential (scrambled control: median=−81.4 mV; GRIN2B shRNA: median=−82.5 mV; ns not significant), (d) input resistance measured with a −20 pA hyperpolarizing step (scrambled control: median=112.8 MΩ; GRIN2B shRNA: median=111.1 MΩ; ns not significant), and (e) rheobase current—current required to evoke first action potential (scrambled control: median=200 pA; GRIN2B shRNA: median=200 pA; ns not significant) between scrambled control and GRIN2B shRNA groups.

FIG. 9A-F. Knocking down GRIN2B has no effect on the forelimb use asymmetry elicited by 6-OHDA and does not imperil the symptomatic benefit of levodopa. Down-regulation of GluN2B containing NMDARs in iSPNs did not change the forelimb use asymmetry produced by 6-OHDA lesioning (GRIN2B shRNA n=6 mice; p>0.05, Wilcoxon test; scrambled control n=6 mice; p>0.05, Wilcoxon test). The reduction in AIM scores had no effect on the anti-parkinsonian benefit of levodopa, as the forelimb use asymmetry was improved by levodopa and GRIN2B shRNA or its scrambled control (levodopa+GRIN2B shRNA n=6 mice, p<0.01, Wilcoxon test; levodopa+scrambled control n=6 mice, p<0.01, Wilcoxon test). **p<0.01, ns not significant.

FIG. 10. Down-regulation of GRIN2B has no effect on the forelimb use asymmetry elicited by 6-OHDA and does not compromise the anti-parkinsonian benefit of levodopa. Knock-down of iSPN GRIN2B did not change the forelimb use asymmetry produced by 6-OHDA lesioning (GRIN2B shRNA n=6 mice; p>0.05, Wilcoxon test; scrambled control n=6 mice; p>0.05, Wilcoxon test). The reduction in AIM scores had no effect on the anti-parkinsonian benefit of levodopa, as the forelimb use asymmetry was improved by levodopa and GRIN2B shRNA or its scrambled control (levodopa+GRIN2B shRNA n=6 mice, p<0.01, Wilcoxon test; levodopa+scrambled control n=6 mice, p<0.01, Wilcoxon test). **p<0.01, ns not significant.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein, the term “effective amount” refers to the amount of a composition (e.g., pharmaceutical composition) sufficient to effect beneficial or desired results. An effective amount can be administered in one or more administrations, applications or dosages and is not intended to be limited to a particular formulation or administration route.

As used herein and in the appended claims, the singular forms “a”, “an” and “the” include plural reference unless the context clearly dictates otherwise. Thus, for example, reference to “a GRIN2B nucleic acid inhibitor” is a reference to one or more a GRIN2B nucleic acid inhibitors, unless the context clearly dictates otherwise.

As used herein, the term “administering” refers to the act of giving a drug, prodrug, or other agent, or therapeutic treatment (e.g., pharmaceutical compositions of the present invention) to a subject or in vivo, in vitro, or ex vivo cells, tissues, and organs. Exemplary routes of administration to the human body can be through the eyes (e.g., intraocularly, intravitreally, periocularly, ophthalmic, etc.), mouth (oral), skin (transdermal), nose (nasal), lungs (inhalant), oral mucosa (buccal), ear, rectal, by injection (e.g., intravenously, subcutaneously, intratumorally, intraperitoneally, etc.) and the like.

As used herein, the terms “co-administration” and “co-administering” refer to the administration of at least two agent(s) (e.g., a GRIN2B nucleic acid inhibitor and one or more additional therapeutics) or therapies to a subject. In some embodiments, the co-administration of two or more agents or therapies is concurrent (e.g., in a single formulation/composition or in separate formulations/compositions). In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “comprise”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for 30 ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

As used herein, the term “shRNA” (small hairpin RNA) refers to an RNA duplex wherein a portion of the RNA is part of a hairpin structure (shRNA). In addition to the duplex portion, the hairpin structure may contain a loop portion positioned between the two sequences that form the duplex. The loop can vary in length. In some embodiments, the loop is 5, 6, 7, 8, 9, 10, 11, 12 or 13 nucleotides in length. The hairpin structure can also contain 3′ or 5′ overhang portions. In some aspects, the overhang is a 3′ or a 5′ overhang 0, 1, 2, 3, 4 or 5 nucleotides in length. In one aspect of this invention, a nucleotide sequence in the vector serves as a template for the expression of a small hairpin RNA, comprising a sense region, a loop region and an antisense region. Following expression, the sense and antisense regions form a duplex. It is this duplex, forming the shRNA, which hybridizes to a target mRNA and reduces expression thereof.

As used herein, the term “knock-down” or “knock-down technology” refers to a technique of gene silencing in which the expression of a target gene or gene of interest is reduced as compared to the gene expression prior to the introduction of the siRNA, which can lead to the inhibition of production of the target gene product.

As used herein, the terms “treatment,” “treating,” and the like refer to obtaining a desired pharmacologic and/or physiologic effect against a particular disease, disorder, or condition. Preferably, the effect is therapeutic, i.e., the effect partially or completely cures the disease and/or adverse symptom attributable to the disease. The terms “treatment” and “treating” refers to reversing, alleviating, slowing down, inhibiting the progression of, preventing, or reducing the likelihood of the disease, disorder, or condition to which such term applies, or one or more symptoms or manifestations of such disease, disorder, or condition. In some embodiments, for example, the terms “treatment” and “treating” of a disease also includes providing relief from the symptoms or side-effects of the disease (including palliative treatment).

As used herein, the term “pharmaceutical composition” refers to the combination of an active agent with a carrier, inert or active, making the composition especially suitable for diagnostic or therapeutic use in vitro, in vivo or ex vivo.

As sed herein, the terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, 10 water, emulsions (e.g., such as an oil/water or water/oil emulsions), and various types of wetting agents, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers, and adjuvants, see, e.g., Martin, Remington's Pharmaceutical Sciences, 15th Ed., 15 Mack Publ. Co., Easton, Pa. (1975), incorporated herein by reference in its entirety.

### DETAILED DESCRIPTION

The present disclosure provides compositions and methods for the treatment of levodopa-induced dyskinesia (LID) therewith. In particular, the present disclosure provides methods for treating LID by knocking down GluN2B in Indirect pathway Spiny Projection Neurons (iSPNs) to attenuate the development and expression of LID without compromising the treatment of levodopa.

Parkinson's disease (PD) is the second most common neurodegenerative disorder. The cardinal motor symptoms of PD—bradykinesia and rigidity or tremor—are caused by loss of functional dopaminergic neurons in the substantia nigra pars compacta (SNc). In the healthy brain, the release of dopamine (DA) by SNc neurons modulates basal ganglia circuitry, promoting movement vigor and goal-directed actions. In the early stages of PD, when a substantial population of SNc dopaminergic neurons remain, boosting their release of DA by administration of its blood-brain barrier penetrant precursor (L-3,4-dihydroxyphenylalanine or levodopa) effectively alleviates motor symptoms. However, as the disease progresses, higher oral doses of levodopa are required to achieve symptomatic benefit and brain concentrations of DA become dysregulated—rising to abnormally high levels for hours and then falling back to very low levels as systemic levodopa wains. These fluctuations lead to uncontrolled, dyskinetic movements when DA levels are high (on-state) and to severely impaired movement when they fall (off-state).

The mechanisms underlying levodopa-induced dyskinesia (LID) are poorly understood and, as a consequence, therapeutic strategies for diminishing their severity are limited. Several lines of evidence point to the importance of aberrant striatal synaptic plasticity in the induction and expression of LID. In the healthy brain, transient alterations in striatal DA signaling in response to action outcomes modulate the induction of corticostriatal synaptic plasticity, altering the probability that the action will be repeated in the future. In late-stage PD patients given high doses of levodopa, not only is the relationship between DA and action outcome severed, but alterations in DA concentration are also sustained for hours. Indeed, during the on-state, when striatal DA levels are high, aberrant enhancement of intrinsic excitability and long-term potentiation (LTP) of corticostriatal synapses on D1 DA receptor (D1R)-expressing, direct pathway spiny projection neurons (dSPNs) appear to play a key role in triggering dyskinetic movements. Consistent with this view, chemogenetic or optogenetic stimulation of dSPNs mimics LID.

Although dSPNs undoubtedly make a significant contribution to the network pathophysiology underlying LID, there are compelling reasons to think that aberrant plasticity in D2 DA receptor (D2R)-expressing indirect pathway SPNs (iSPNs) also contributes. Normally, iSPNs are thought to promote context appropriate action by suppressing inappropriate, competing actions. This learned activity is widely thought to depend upon DA-driven adjustments in the strength of iSPN corticostriatal glutamatergic synapses. Unlike the situation in dSPNs, DA signaling (associated with reward) triggers long-term depression (LTD) of active iSPN corticostriatal synapses, presumably lessening their ability to suppress appropriate actions. In contrast, dips in DA signaling associated with negative outcomes disinhibits iSPN adenosine 2a receptors (A2aRs), promoting LTP of active corticostriatal synapses, which presumably leads to a ‘veto’ of that action in the future. This precisely timed, action-outcome-linked sculpting of iSPN synapses is not restored by levodopa treatment in late-stage PD patients lacking SNc dopaminergic neurons. Rather, these mechanisms appear to be hijacked for hours following levodopa treatment, driving iSPN synaptic plasticity in one direction during the on-state (down) and then in the opposite direction (up) during the off-state—all independently of behavioral outcomes.

Precisely how these alternating periods of plasticity remodel iSPNs is unclear. Several reports have highlighted the potential importance of alterations in N-methyl-d-aspartate receptors (NMDARs) at striatal glutamatergic synapses in LID. As at other glutamatergic synapses, NMDARs are necessary for the induction of LTP at corticostriatal synapses. However, it is uncertain whether any of these changes are happening specifically in iSPNs and, if so, whether they impact dyskinetic behavior. A clue that these synaptic processes could be important is that dyskinesiogenic doses of levodopa trigger the restoration of iSPN axospinous glutamatergic synapses, which are lost following DA depletion. Using a combination of electrophysiological, genetic, pharmacological, and behavioral approaches, the studies described here show that dyskinesiogenic doses of levodopa up-regulate GluN2B-containing NMDARs in iSPNs, but not in dSPNs. This up-regulation leads to a significant elevation in the abundance of ‘silent’ glutamatergic synapses in the off-state and the induction of A2aR-dependent LTP. Connecting these cellular changes to behavior, A2aR antagonism in the off-state reduced the induction of LID. In addition, selectively suppressing expression of the GluN2B subunit in iSPNs significantly diminished not only the induction of LID, but the expression of established LID. Taken together, these studies provide a fundamental insight into mechanisms underlying LID and point to novel therapeutic strategies to alleviate them.

Embodiments of the present disclosure include a method for treating Levodopa-induced dyskinesia (LID), in a subject in need thereof, the method comprising: administering a GRIN2B inhibitor to the subject.

In some embodiments, the GRIN2B inhibitor is administered into an indirect pathway spiny projection neuron (iSPN). In some embodiments, the GRIN2B inhibitor is administered systemically.

In some embodiments, the GRIN2B inhibitor reduces GluN2B protein levels.

In some embodiments, the GRIN2B inhibitor is an inhibitor of GRIN2B expression.

In some embodiments, the GRIN2B inhibitor is a nucleic acid inhibitor of GRIN2B expression.

In some embodiments, the GRIN2B inhibitor knocks down the expression of GluN2B protein to suppress the induction of iSPN adenosine 2a receptors (A2aRs)-dependent long-term potentiation (LTP).

In some embodiments, the nucleic acid inhibitor is a microRNA (miRNA), a small interfering (siRNA), a short hairpin RNA (shRNA), an anti-sense RNA (asRNA), a competing endogenous RNA (ceRNA), a long non-coding RNA (lncRNA) and a ribozyme.

In some embodiments, the nucleic acid inhibitor is dispersed into a vector. In other embodiments, the vector is a viral vector. In further embodiments, the viral vector is an adeno-associated viral vector (AAV), a adenoviral vector, a lentiviral vector, a non-viral vector, a human-compatible vector, a herpes simplex virus vector and a retroviral vector.

In some embodiments, a siRNA is an 18 to 30 nucleotide, preferably 19 to 25 nucleotide, most preferred 21 to 23 nucleotide or even more preferably 21 nucleotide-long double-stranded RNA molecule. siRNA is involved in the RNA interference (RNAi) pathway where the siRNA interferes with the expression of a specific gene (e.g., GRIN2B). siRNAs naturally found in nature have a well-defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest (e.g., GRIN2B). Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, in some embodiments at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA and inhibit GRIN2B is envisioned in the present invention. In some embodiments, siRNA duplexes are provided composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair. 2′-deoxynucleotides in the 3′ overhangs are as efficient as ribonucleotides but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as 5% glucose) or cationic lipids and polymers can be used for siRNA delivery in vivo through systemic routes either intravenously (IV) or intraperitoneally (IP).

In some embodiments, the inhibitory nucleic acid (i.e., GRIN2B inhibitor) is a short hairpin RNA (shRNA). An shRNA is a sequence of RNA that makes a tight hairpin turn that can be used to silence gene expression (e.g., of GRIN2B) via RNA interference. In some embodiments, si/shRNAs to be used in the present invention are chemically synthesized using appropriately protected ribonucleoside phosphoramidites and a conventional DNA/RNA synthesizer. In some embodiments, provided herein are shRNA molecules that target and inhibit the expression (e.g., knockdown) of the GRIN2B.

In experiments conducted during development of embodiments herein, a shRNA having 100% identify to SEQ ID NO: 1 (GCTGGTGATAATCCTTCTGAA) was used to demonstrate inhibition of GRIN2B expression by a nucleic acid inhibitor of GRIN2B as well as treatment and prevention of LID therewith. In some embodiments, provided herein are shRNA molecules having at least 70% (e.g., 70%, 75%, 80%, 85%, 90%, 95%, 100%, or ranges therebetween) sequence identity to SEQ ID NO: 1. In some embodiments, methods are provided for the treatment or prevention of PD and/or LID comprising the administration of an shRNA molecule having at least 70% (e.g., 70%, 75%, 80%, 85%, 90%, 95%, 100%, or ranges therebetween) sequence identity to SEQ ID NO: 1.

An exemplary shRNA having 100% identity to CTTCAGTGAAGATGGCTACCA (SEQ ID NO: 2) is useful in inhibition of GRIN2B expression in both human and mice, and therefore may find use as both a therapeutic/prophylactic (e.g., in humans) and a research tool (e.g., in humans and mice). In some embodiments, provided herein are shRNA molecules having at least 70% (e.g., 70%, 75%, 80%, 85%, 90%, 95%, 100%, or ranges therebetween) sequence identity to SEQ ID NO: 2. In some embodiments, methods are provided for the treatment or prevention of PD and/or LID comprising the administration of an shRNA molecule having at least 70% (e.g., 70%, 75%, 80%, 85%, 90%, 95%, 100%, or ranges therebetween) sequence identity to SEQ ID NO: 2.

Further molecules effecting RNAi (and useful herein for the inhibition of expression of the GRIN2B) include, for example, microRNAs (miRNA). Said RNA species are single-stranded RNA molecules. Endogenously present miRNA molecules regulate gene expression by binding to a complementary mRNA transcript and triggering the degradation of said mRNA transcript through a process similar to RNA interference. Accordingly, exogenous miRNA may be employed as an inhibitor of GRIN2B after introduction into target cells. In some embodiments, provided herein are miRNA molecules that target and inhibit the expression (e.g., knock down) of the GRIN2B.

Morpholinos (or morpholino oligonucleotides) are synthetic nucleic acid molecules having a length of about 20 to 30 nucleotides and, typically about 25 nucleotides. Morpholinos bind to complementary sequences of target transcripts (e.g., GRIN2B) by standard nucleic acid base-pairing. They have standard nucleic acid bases which are bound to morpholine rings instead of deoxyribose rings and linked through phosphorodiamidate groups instead of phosphates. Due to replacement of anionic phosphates into the uncharged phosphorodiamidate groups, ionization in the usual physiological pH range is prevented, so that morpholinos in organisms or cells are uncharged molecules. The entire backbone of a morpholino is made from these modified subunits. Unlike inhibitory small RNA molecules, morpholinos do not degrade their target RNA molecules. Rather, they sterically block binding to a target sequence within a RNA and prevent access by molecules that might otherwise interact with the RNA. In some embodiments, provided herein are morpholino oligonucleotides that target and inhibit the expression (e.g., knockdown) of GRIN2B.

A ribozyme (ribonucleic acid enzyme, also called RNA enzyme or catalytic RNA) is an RNA molecule that catalyzes a chemical reaction. Many natural ribozymes catalyze either their own cleavage or the cleavage of other RNAs, but they have also been found to catalyze the aminotransferase activity of the ribosome. Non-limiting examples of well-characterized small self-cleaving RNAs are the hammerhead, hairpin, hepatitis delta virus, and in vitro-selected lead-dependent ribozymes, whereas the group I intron is an example for larger ribozymes. The principle of catalytic self-cleavage is well established. Since it was shown that hammerhead structures can be integrated into heterologous RNA sequences and that ribozyme activity can thereby be transferred to these molecules, catalytic antisense sequences can be engineered for almost any target sequence that can be created, provided the target sequence contains a potential matching cleavage site. The basic principle of constructing hammerhead ribozymes is as follows: A region of interest of the RNA (e.g., a portion of GRIN2B), which contains the GUC (or CUC) triplet, is selected. Two oligonucleotide strands, each usually with 6 to 8 nucleotides, are taken and the catalytic hammerhead sequence is inserted between them. In some embodiments, provided herein are ribozyme inhibitors oligonucleotides of the GRIN2B.

In some embodiments, the gene-silencing siRNA is a GRIN2B siRNA. In some embodiments, the gene-silencing siRNA is capable of targeting an mRNA encoding GRIN2B and inhibiting the translation of GRIN2B.

In some embodiments, provided herein are inhibitors of GRIN2B expression, for example, nucleic acid inhibitors of GRIN2B (e.g., shRNA, antisense RNA, siRNA, etc.) capable of binding to a portion of the GRIN2B gene (SEQ ID NO: 3) and/or inhibiting GRIN2B expression by the mechanisms described herein.

In some embodiments, the subject is afflicted with the neurodegenerative disease associated with an upregulation of GluN2B-containing N-methyl-D-aspartate receptors (NMDARs).

In some embodiments, the neurodegenerative disease from Parkinson's disease.

In some embodiments, the subject is a human. In some embodiments, the subject was administered or is currently being a therapeutic agent.

In some embodiments, the therapeutic agent administered is levodopa (L-3,4-dihydroxyphenylalanine).

Levodopa (L-DOPA) or L-3,4-dihydroxyphenylalanine: is a chemical that is made and used as part of the normal biology of humans, some animals, and plants. Levodopa is the precursor to the neurotransmitters dopamine, norepinephrine (noradrenaline), and epinephrine (adrenaline) collectively known as catecholamines. Levodopa mediates neurotrophic factor release by the brain and CNS. Levodopa is manufactured and sold as a psychoactive drug under trade names including SINEMET, PHARMACOPA, ATAMET, STALEVO, MADOPAR, and PROLOPA. Levodopa is prescribed and administered for the treatment of neurodegenerative disorders, and in particular in the clinical treatment of Parkinson's disease.

In other embodiments, administering levodopa to the subject induces fluctuations of dopamine (DA) levels in a subject; wherein large and uncontrolled fluctuations in DA levels results in levodopa-induced dyskinesia (LID).

In some embodiments, levodopa is initially administered to a subject at a dose of 200-600 mg. In some embodiments, levodopa is initially administered to a subject at a dose of 200 mg. In some embodiments, levodopa is initially administered to a subject at a dose of 250 mg. In some embodiments, levodopa is initially administered to a subject at a dose of 300 mg. In some embodiments, levodopa is initially administered to a subject at a dose of 350 mg. In some embodiments, levodopa is initially administered to a subject at a dose of 400 mg. In some embodiments, levodopa is initially administered to a subject at a dose of 450 mg. In some embodiments, levodopa is initially administered to a subject at a dose of 500 mg. In some embodiments, levodopa is initially administered to a subject at a dose of 550 mg. In some embodiments, levodopa is initially administered to a subject at a dose of 600 mg.

In some embodiments, levodopa is administered to a to a subject once a day. In some embodiments, levodopa is administered to a to a subject twice a day. In some embodiments, levodopa is administration is increased over time.

In some embodiments, levodopa is administered to a subject at a maintained dose of 2000 to 7000 mg/day. In some embodiments, levodopa is administered to a subject at a maintained dose of 2000 mg/day. In some embodiments, levodopa is administered to a subject at a maintained dose of 2500 mg/day. In some embodiments, levodopa is administered to a subject at a maintained dose of 3000 mg/day. In some embodiments, levodopa is administered to a subject at a maintained dose of 3500 mg/day. In some embodiments, levodopa is administered to a subject at a maintained dose of 4000 mg/day. In some embodiments, levodopa is administered to a subject at a maintained dose of 4500 mg/day. In some embodiments, levodopa is administered to a subject at a maintained dose of 5000 mg/day. In some embodiments, levodopa is administered to a subject at a maintained dose of 5500 mg/day. In some embodiments, levodopa is administered to a subject at a maintained dose of 6000 mg/day. In some embodiments, levodopa is administered to a subject at a maintained dose of 6500 mg/day. In some embodiments, levodopa is administered to a subject at a maintained dose of 7000 mg/day.

In some embodiments, levodopa is administered to a subject at a maintained dose into three separate dosages. In some embodiments, levodopa administration does not exceed a dose of 8000 mg/day.

In further embodiments, the administering levodopa to the subject increases the expression of the GluN2B-containing NMDARs in iSPNs. In some embodiments, the increased expression of the GluN2B-containing NMDARs in iSPNs during fluctuations of DA levels results in the upregulation of glutamatergic synapses and the induction of A2aR-dependent LTP.

In some embodiments, the GRIN2B inhibitor knocks down the expression of GluN2B to suppress the induction of iSPN adenosine 2a receptors (A2aRs)-dependent long-term potentiation (LTP) to diminish the induction and expression of levodopa-induced dyskinesia (LID).

In some embodiments, the GRIN2B inhibitor is an Intraperitoneal (IP) injection.

Provided herein are compositions and methods treating PD with levodopa and an GRIN2B inhibitor.

In some embodiments, said GRIN2B inhibitor is administered repeatedly to the subject. In other embodiments, said GRIN2B inhibitor is administered once to the subject.

In some embodiments, wherein said GRIN2B inhibitor is administered during the low levels of DA to the subject after levodopa is administered.

In some embodiments, the use of GRIN2B inhibitor for use in the manufacture of a medicament for the treatment of a neurodegenerative disease in a subject in need thereof.

In some embodiments, the use of GRIN2B inhibitor for treatment of a neurodegenerative disease in a subject in need thereof.

In some embodiments, the use of GRIN2B inhibitor for use in the manufacture of a medicament for the treatment of Levodopa-induced dyskinesia (LID) in a subject in need thereof.

In some embodiments, the use of GRIN2B inhibitor for use in the treatment of Levodopa-induced dyskinesia (LID) in a subject in need thereof.

As is well known in the medical arts, dosages (e.g., levodopa dosages, etc.) for any one patient depends upon many factors, including the patient's size, body surface area, age, the particular compound to be administered, sex, time and route of administration, general health, and interaction with other drugs being concurrently administered.

Pharmaceutical compositions herein may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, pharmaceutical compositions may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions are formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions include compositions wherein the active ingredients (e.g., levodopa, etc.) are contained in an effective amount to achieve the intended purpose. For example, an effective amount of therapeutic may be an amount that prevents LID and/or treats or reduces symptoms associated with PD. Determination of effective amounts is well within the capability of those skilled in the art, especially in light of the disclosure provided herein.

In addition to the active therapeutic ingredients, pharmaceutical compositions may contain suitable pharmaceutically acceptable carriers comprising excipients and auxiliaries that facilitate processing of the active compounds into preparations that can be used pharmaceutically. The preparations formulated for oral administration may be in the form of tablets, dragees, capsules, or solutions. The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropyl methyl-cellulose, or sodium carboxymethylcellulose; and gums including arabic and tragacanth; and proteins such as gelatin and collagen. If desired, disintegrating, or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (e.g., dosage).

Pharmaceutical preparations for oral administration include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Therapeutic compositions formulated in a pharmaceutical acceptable carrier may be prepared, placed in an appropriate container, and labeled for treatment of the indicated condition (e.g., levodopa-induced dyskinesias, PD, etc.).

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

In some embodiments, a therapeutically effective dose may be estimated initially from cell culture assays and/or animal models (particularly murine models). A therapeutically effective dose refers to that amount that effectively addresses and underlying cause and/or ameliorates symptoms of the disease state or unwanted condition (e.g., levodopa-induced dyskinesias, PD, etc.). Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. Data obtained from these cell culture assays, and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration. The exact dosage is chosen by the individual clinician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination (s), reaction sensitivities, and tolerance/response to therapy. Long-acting pharmaceutical compositions might be administered every 3 to 4 days, every week, or once every two weeks depending on half-life and clearance rate of the particular formulation.

Typical dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; 5,225,212; WO2004/097009, or WO2005/075465, each of which are herein incorporated by reference).

In some embodiments, the therapies disclosed herein are combined or used in combination with other agents useful in the treatment of psychomotor diseases (e.g., PD). Or, by way of example only, the therapeutic effectiveness of one of the therapies described herein may be enhanced by administration of an adjuvant (e.g., by itself the adjuvant may only have minimal therapeutic benefit, but in combination with another therapeutic agent, the overall therapeutic benefit to the patient is enhanced).

Such other agents, adjuvants, or drugs, may be administered, by a route and in an amount commonly used therefor, simultaneously or sequentially with a compound as disclosed herein. When a compound as disclosed herein is used contemporaneously with one or more other drugs, a pharmaceutical composition containing such other drugs in addition to the compound disclosed herein may be utilized but is not required.

In some embodiments, one or more of the therapies provided herein (e.g., levodopa, etc.) are combined with each other, and/or with one or more treatments for a psychomotor disease (e.g., PD). Suitable treatments for psychomotor disease (e.g., PD) for co-administration (e.g., with FRIN2B inhibitor, levodopa, etc.) include dopamine agonists (e.g., bromocriptine, pergolide, pramipexole, ropinirole, piribedil, cabergoline, apomorphine, lisuride, etc.), MAO-B inhibitors (e.g., selegiline, rasagiline, etc.), and other therapeutics, such as amantadine, anticholinergics, quetiapine, cholinesterase inhibitors, modafinil, non-steroidal anti-inflammatory drugs, etc.

In some embodiments, deep brain stimulation is also utilized for the treatment of PD, in addition to the other embodiments described herein.

In some embodiments, one or more therapeutic approaches described herein co-administered to a subject. In some embodiments, co-administration involves co-formulation of two or more agents together into the same medicament. In other embodiments, the agents are in separate formulations but are administered together, either simultaneously or in sequence (e.g., separated by one or more minutes, hours, days, etc.). In some embodiments, where a synergistic or additive benefit is achieved, the co-administered agent may be provided at a lower dose than would normally be administered if that agent were being used in isolation to treat the disease or condition.

### EXPERIMENTAL

The following Materials/Methods and Examples are offered as illustrative as a partial scope and particular embodiments of the disclosure and are not meant to be limiting of the scope of the disclosure.

### Animals

Male C57Bl/6 mice expressing tdTomato or eGFP under control of either the Drd1a or Drd2 receptor regulatory elements (NINDS GENSAT BAC Transgenics Project, Rockefeller University, New York, NY) were used. All mice used for were hemizygous for these transgenes. BAC transgenic mice expressing Cre recombinase under control of the A2aR regulatory elements were used for GRIN2B shRNA experiments. All mice were 8-10 weeks of age before stereotaxic surgery.

### Slice Preparation and Electrophysiology

Mice were deeply anesthetized intraperitoneally with a mixture of ketamine (50 mg kg-1) and xylazine (4 mg kg-1) and perfused transcardially with 5-10 ml of ice-cold artificial CSF (aCSF) comprising (in mM): 125 NaCl, 2.5 KCl, 1.25 NaH2PO4, 2.0 CaCl2), 1.0 MgCl2, 26 NaHCO₃ and 10 glucose (305 mOsm 1-1). Parasagittal slices were cut in ice-cold external solution containing (mM) 110 choline chloride, 26 NaHCO₃, 1.25 NaH2PO4, 2.5 KCl, 0.5 CaCl2), 7 MgCl2, 11.6 sodium ascorbate, 3.1 sodium pyruvate and 5 glucose (305 mOsm 1-1). Experiments were performed in the dorsolateral striatum at elevated temperature (30-31° C.). Patch pipettes were loaded with internal solution containing (mM): 120 CsMeSO3, 15 CsCl, 8 NaCl, 10 HEPES, 0.2 EGTA, 10 TEA Chloride, 5 QX-314, 2 Mg-ATP, 0.3 Na-GTP (305 mOsm 1-1) for whole-cell voltage-clamp recordings; or 115 K-gluconate, 20 KCl, 1.5 MgCl2, 5 HEPES, 0.2 EGTA, 2 Mg-ATP, 0.5 Na-GTP, 10 Na-phosphocreatine (280 mOsm 1-1) for whole-cell current-clamp recordings. All the recordings were made using a MultiClamp 700B amplifier, and signals were filtered at 2 kHz and digitized at 10 kHz. Data were discarded when the series resistance (voltage-clamp) or input resistance (current-clamp) changed >20% over the time course of the experiment. For perforated patch recordings, the internal recording solution comprised (in mM): 125 KmeSO4, 14 KCl, 2 MgCl2, 0.2 EGTA, 10 HEPES. Amphotericin B was used to achieve electrical access through the perforated-patch method.

### Mouse Unilateral 6-OHDA Model and LID

Mice were anaesthetized with an isoflurane precision vaporizer, placed in a stereotaxic frame, and a hole was drilled over the MFB. After exposing the skull, 3.5 mg ml-1 free base 6-OHDA hydrochloride with 0.02% ascorbic acid was injected using a calibrated glass micropipette at the following coordinates: AP: −0.7; ML: −1.2; DV: −4.75. Two-three weeks after surgery, the degree of damage to nigrostriatal DA neurons was assessed with a forelimb-use asymmetry test. Well-lesioned animals were then assigned to receive either GRIN2B shRNA or its scrambled control in the DLS in a total of 6 sites at the following coordinates: AP: 0.9; ML: −2.3; DV: −3.4; −2.8; AP: 0.6; ML: −1.5; DV: −3.4; −2.8; AP: 0.24; ML: −1.9; DV: −3.3; −2.7. One day after the forelimb-use asymmetry test, mice underwent behavioral testing for AIMs following levodopa treatment as previously described. For ex vivo brain slice recording, animals received daily levodopa for either short (3-5 days) or prolonged period (12-14 days). Benserazide was co-administered at 12 mg kg-1 to inhibit peripheral conversion of levodopa to DA. AIMs (axial, limb and orolingual movements) were rated. Each animal was observed individually for 1 minute every 20 minutes for 2 hours. Physiological experiments were performed one day (off-state) or one hour (on-state) after the last levodopa administration.

### Immunostaining and Confocal Microscopy

After behavioral experiments, mice that had been injected with AAV-DIO-GRIN2B shRNA/GFP were anesthetized as described above and perfused transcardially with saline for ˜1 minute and then with ice-cold 4% paraformaldehyde (wt/vol) in 1× phosphate buffered saline (4% PFA-PBS). The mouse brains were dissected out, post-fixed in 4% PFA-PBS overnight at 4° C., and sectioned into coronal slices (100 μm-thick) using a Leica vibratome (VT1200, Leica). Immunostaining was performed. Briefly, the fixed brain slices were permeabilized and blocked in PBS containing 5% normal goat serum and 0.2% Triton-X100 (NGS-PBST) for 1 hour at 4° C., and then incubated with mouse monoclonal anti-NMDAR2B antibody (1:100 dilution in NGS-PBST, ab93610, Abcam) overnight at 4° C. After four washes in NGS-PBST, the slices were incubated with goat anti-mouse Alexa Fluor 555 secondary antibody (1:1000 dilution in NGS-PBST, A-21422, Invitrogen) for 2 hours at room temperature. Slices were washed with NGS-PBST four times, counter-stained with DAPI (1:1000 dilution in PBS, D1306, Invitrogen), and washed again with PBS once. Slices were then mounted with VECTASHIELD Mounting Medium (Vector Laboratories) and imaged under a laser scanning confocal microscope (FV10i-DUC; Olympus). Images were adjusted for brightness, contrast, and pseudo-coloring in ImageJ (US National Institutes of Health).

### Viral Vectors

The shRNA sequence targeting GRIN2B mRNA was inserted into the miR30 backbone of LENG plasmid, (Addgene plasmid #111162) and then into 3′-UTR of GFP in pAAV-Ef1a-DIO GFP plasmid, (Addgene plasmid #27056). YFP was substituted for GFP in this construct. The hairpin for the miR30-based expression of scrambled shRNA, (Addgene plasmid #71383) was inserted into 3′-UTR of GFP in pAAV-Ef1a-DIO GFP plasmid in similar way. Viruses were packaged by Virovek.

### Data Analysis and Statistics Methods

Data analysis was conducted with Igor Pro 8 and Clampfit 10. EPSP amplitude was calculated from 50 sweeps immediately before the start of induction and 20-30 minutes after the end of induction. Compiled data were expressed as mean±SEM. Statistical tests were performed using Excel and SigmaStat. Nonparametric Mann-Whitney rank sum and Wilcoxon signed rank tests were used to assess the experiment results, using a probability threshold of 0.05. Statistical analysis for behavioral data was carried out using Prism 10. Data were analyzed using parametric repeated measure two-way ANOVA followed by post hoc Fisher or Bonferroni's test.

### Example 1

### LID Induction was Correlated with an Up-Regulation of GluN2B-Containing NMDARs in iSPNs

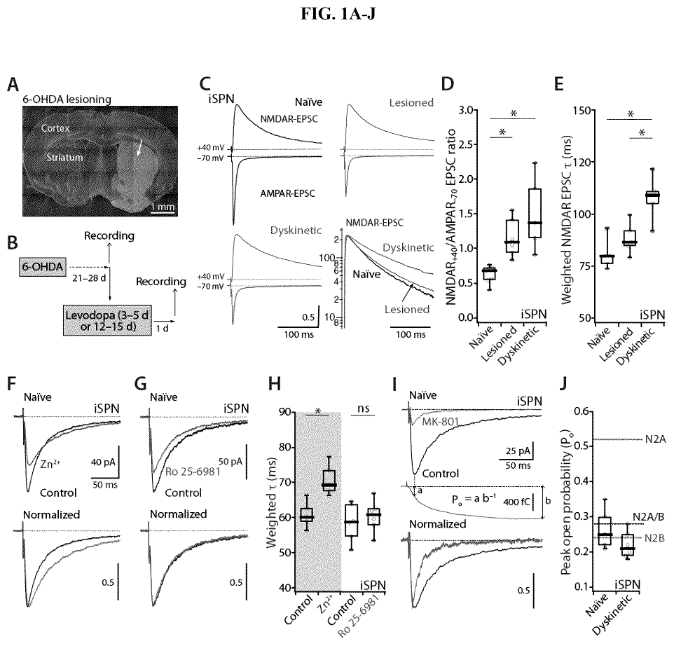

Electrophysiological and pharmacological methods were used to assess the relative contribution of α-amino-3-hydroxy-5-methyl-4-isoxazolepropionic acid receptors (AMPARs) and NMDARs to iSPN glutamatergic, excitatory postsynaptic currents (EPSCs) in ex vivo brain slices from naive, 6-hydroxydopamine (6-OHDA) lesioned, and 6-OHDA-lesioned mice induced to express dyskinesia by repeated administration of levodopa. Briefly, Drd2-eGFP bacterial artificial chromosome (BAC) transgenic mice were subjected to unilateral medial forebrain bundle (MFB) injections of 6-OHDA (FIG. 1a). Three to four weeks later, the extent of the lesions was assessed using a forelimb-use asymmetry test (also called cylinder test). Mice with a robust lesion [>85% striatal tyrosine hydroxylase (TH) loss] were given either a short (3-5 days) or long (12-14 day) regimen of levodopa at a dose inducing dyskinesia in all animals (3 mg/kg/day, i.p.) (FIG. 1b). Mice were then sacrificed, striatal brain slices prepared and dorsolateral striatal (DLS) iSPNs patch clamped in the presence of the GABAA receptor antagonist gabazine (10 μM). The relative contribution of AMPARs and NMDARs to electrically evoked EPSCs was estimated by measuring current amplitudes at −70 mV and then +40 mV in the presence of an AMPAR antagonist NBQX (10 μM) (FIG. 1c, d). In iSPNs from untreated, 6-OHDA lesioned mice, the NMDAR/AMPAR ratio was elevated (FIG. 1c, d); this shift was attributable to a reduction in AMPAR abundance. The elevation in the NMDAR/AMPAR ratio was also evident in iSPNs from lesioned mice treated with dyskinesiogenic doses for either short (3-5 days) or long periods (12-14 days) (FIG. 1c, d). However, NMDAR currents in the levodopa-treated iSPNs decayed significantly slower than those in untreated iSPNs (FIG. 1c, e).

NMDARs are tetramers, consisting of two GluN1 subunits and two GluN2 subunits. In adult forebrain neurons, the majority of NMDARs are di- and tri-heteromers of GluN2A and/or 2B subunits. NMDARs with GluN2B subunits deactivate more slowly than those with only GluN2A subunits, indicating that the slowing of NMDAR currents following levodopa treatment is a manifestation of an increasing contribution of GluN2B-containing NMDARs to synaptic currents.

Pharmacological tools were used to assess the relative contribution of GluN2A- and GluN2B-containing NMDARs to evoked currents in iSPNs. In ex vivo brain slices from naive mice, bath application of the GluN2A-preferring inhibitor Zn2+ (100 μM with 10 mM tricine) reduced the amplitude of NMDAR-mediated currents and slowed their de-activation 36, 43 (FIG. 1f, h). Bath application of the GluN2B inhibitor Ro 25-6981 (1 μM) also reduced the amplitude of the NMDAR currents but did not alter their deactivation kinetics (FIG. 1g, h). This observation is consistent with work in hippocampal neurons showing that the GluN2A subunit determines NMDAR channel deactivation kinetics.

To gain more insight into the contribution of GluN2A and GluN2B subunits to synaptic NMDARs, the peak open probability (Po) was estimated using the high-affinity NMDAR open channel blocker MK-801 (25 μM) 36, 44. To estimate Po, the ratio of charge transfer at the peak of the EPSC (a) to the total charge transfer (b) was computed in the presence of MK-801 (Po=a/b) (FIG. 1i) 36. Because GluN2A-containing NMDARs activate more rapidly than GluN2B-containing ones, the Po measured at the peak of the EPSC is correlated with subunit composition, being near 0.5 for di-heteromeric GluN1/GluN2A channels and roughly half that for di-heteromeric GluN1/GluN2B channels (FIG. 1j) 36. Furthermore, the Pos of tri-heteromeric GluN1/GluN2A/2B channels is similar to that of di-heteromeric GluN1/GluN2B receptors, indicating that GluN2B subunit determines NMDAR channel opening kinetics. The Pos of iSPN NMDARs from naive and dyskinetic animals fell near the value of di-heteromeric GluN1/GluN2B or tri-heteromeric GluN1/GluN2A/B receptors (FIG. 1i, j), indicating that synaptic NMDARs in iSPNs were largely GluN2B-containing tri- or diheteromeric receptors.

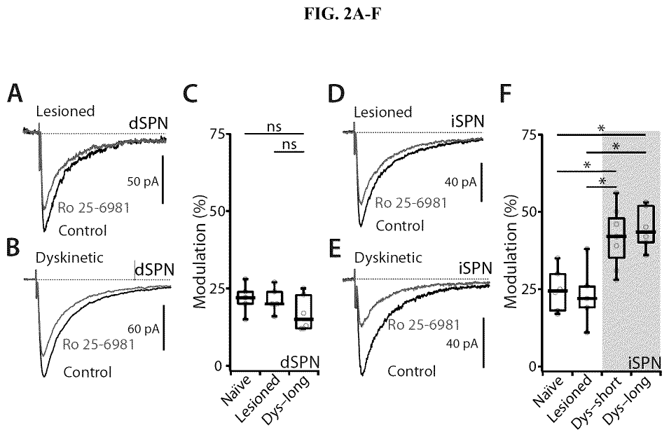

To provide another assessment of the contribution of GluN2B-containing channels to synaptic NMDARs in different states, the ability of the GluN2B-selective antagonist Ro 25-6981 (1 μM) to reduce currents was examined in ex vivo brain slices from naive, 6-OHDA lesioned and dyskinetic mice. Both Drd1-tdTomato and Drd2-eGFP mice were used in these experiments to allow the cellular specificity of effects to be determined. In dSPNs, the contribution of GluN2B-containing NMDARs to the synaptic currents was similar in each of the three conditions (FIG. 2a, b, c). This was not the case for iSPN, however. In iSPNs from naive and 6-OHDA lesioned mice, the percent reduction in EPSC amplitude produced by Ro 25-6981 was roughly 25% (FIG. 2d, e, f); however, in iSPNs from dyskinetic mice, the contribution of GluN2B-containing NMDARs was about 40% (FIG. 2d, e). This change was similar in mice treated with levodopa for 3-5 or 12-14 days (FIG. 2f).

These results demonstrate that with LID induction, there is an up-regulation in GluN2B-containing NMDARs at iSPN glutamatergic synapses, but not at dSPN glutamatergic synapses. Given that in naive iSPNs synaptic NMDARs appear to be largely (if not entirely) GluN2B-containing (based upon the Po estimates), the up-regulation in the contribution of GluN2B subunits in the dyskinetic state could reflect an increased abundance of di-heteromeric GluN1/GluN2B NMDARs at iSPN glutamatergic synapses.

### Example 2

### LID Induction was Correlated with an Increased Abundance of iSPN Silent Synapses

Following 6-OHDA lesioning, spine density in iSPNs falls by roughly a third, presumably reflecting a form of homeostatic plasticity that accompanies the loss of inhibitory D2R signaling. With repeated treatment at dyskinesiogenic doses of levodopa, iSPN spine density returns into a normal range when measured after levodopa has cleared (off-state).

Thus, it is possible that the up-regulation in the contribution of GluN2B-containing NMDARs to evoked currents following LID induction reflects the addition of new axospinous synapses, rather than a modification of pre-existing synapses. In the developing brain, many glutamatergic synapses are dominated by GluN2B di-heteromeric NMDARs and have relatively few AMPARs, leading to ‘silent’ synapses that don't pass current at resting membrane potentials (˜−70 mV) where NMDARs are blocked by Mg2+ 32, 45.

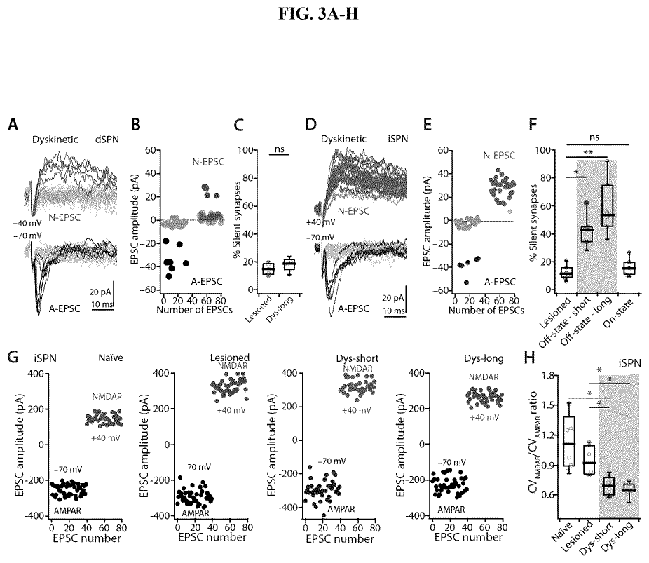

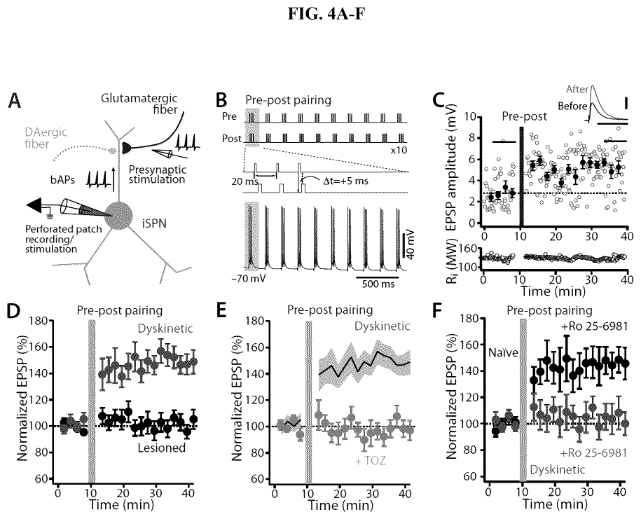

To determine whether SPNs up-regulate AMPA-deficient, silent synapses in the dyskinetic state, two different assays were employed. The first approach used minimal local stimulation to activate a small, random population of glutamatergic synapses and whole-cell voltage clamp methods to monitor postsynaptic currents. Given the stochastic nature of synaptic transmission, each round of stimulation in this protocol evokes glutamate release at some synapses, but not others. For the postsynaptic cell to detect release, glutamate receptors must be present to generate detectable currents. If some of the synapses lack AMPARs but do have NMDARs, then the frequency of failures (where there is no detectable evoked current) will be voltage-dependent: that is, at membrane potentials where NMDARs are blocked by Mg2+ (˜−70 mV) the failure rate will be higher than at membrane potentials where NMDARs can report glutamate release (˜+40 mV). If it is assumed that the presynaptic release sites are independent and the release probabilities are the same at all the synapses, then the percentage of silent synapses can be estimated using the equation: 1−[ln(F−70)/ln(F+40)], where F−70 is the failure rate at −70 mV and F+40 is the failure rate at +40 mV 46. This assay indicates that the percentage of dSPN silent synapses was not discernibly different in 6-OHDA lesioned and LID mice (˜20%, FIG. 3a-c). However, in iSPNs from LID mice the percentage of silent synapses was significantly higher after a 3-5 days or 12-14 days treatment of levodopa (˜40-50%, FIG. 3d-f).

These measurements were all taken in mice well after the last dose of levodopa, mimicking the off-state. To assess whether this change was state-dependent, the minimal local stimulation experiments were repeated in ex vivo brain slices taken from dyskinetic mice an hour after levodopa treatment. In on-state iSPNs, the relative abundance of silent synapses was no different than that in levodopa-naive, 6-OHDA lesioned mice and significantly less than that in the off-state (FIG. 3f). Hence, the change in iSPN synaptic function was state-dependent.

To complement the failure rate assay, the relative variability of EPSCs was examined. In general, the coefficient of variation (CV) of EPSCs is inversely related to the number of contributing synapses. If there were more silent synapses, then the CV of NMDAR EPSCs (CVNMDAR) should be lower than that of AMPAR EPSCs (CVAMPAR). To test this hypothesis, the CV of NMDAR EPSCs and AMPAR EPSCs was measured. After either a short or long-term treatment of levodopa, the CVNMDAR/CVAMPAR ratio fell in iSPNs (FIG. 3g, h).

### Example 3

### Up-Regulation in GluN2B NMDARs Enhanced LTP in iSPNs

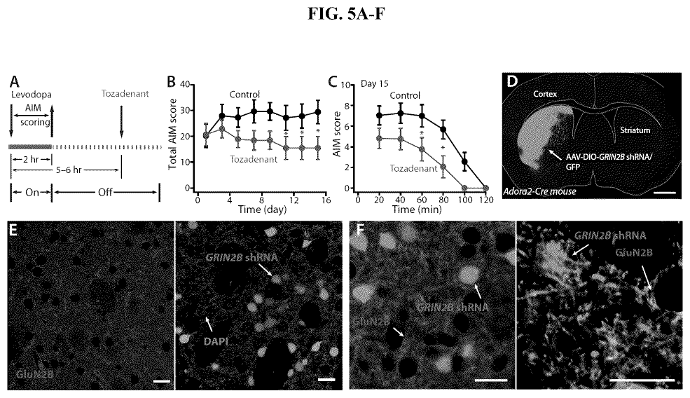

In iSPNs, the induction of spike-timing dependent LTP relies upon the coordinated activation of NMDARs and A2a-Rs. Given their well-established linkage to LTP induction, the up-regulation in GluN2B-containing NMDARs in iSPNs from dyskinetic mice should promote LTP induction, particularly in the LID off-state when D2R signaling is low and A2aR signaling is nominally high. To test this hypothesis, iSPNs from off-state LID mice were subjected to perforated patch, current-clamp recording in ex vivo brain slices and a pre-post, spike timing dependent plasticity (STDP) induction protocol was applied (FIG. 4a, b). In this protocol, afferent fibers were stimulated in short bursts (3 stimuli) with a theta electrode placed within approximately 100 microns from the cell body and trailing back-propagating action potentials (bAPs) (delay 5 ms) generated by short (1000 msec), intrasomatic current pulses. The pairing protocol was repeated every 200 msec for 2 seconds. This pairing protocol resulted in a robust LTP of glutamatergic EPSPs in naive iSPNs (FIG. 4c, d). In iSPNs from levodopa-naive, 6-OHDA lesioned mice, this protocol failed to produce any significant change in EPSP amplitude (FIG. 4d). However, STDP LTP was induced in iSPNs from LID mice subjected to short-term (3-5 days) (FIG. 4d) or long-term (12-14 days) (Supplementary FIG. 1) systemic administration of levodopa. Pharmacological tools were used to assess the role A2aRs and GluN2B-containing NMDARs in the induction of STDP LTP. As expected, bath application of the A2aR antagonist tozadenant (1 μM) prevented LTP induction (FIG. 4e). Similarly, bath application of GluN2B antagonist Ro 25-6981 blunted STDP LTP induction in iSPNs from LID mice in the off-state (FIG. 4f). In brain slices from naive mice, inhibiting GluN2B containing NMDARs had no effect on pre-post STDP LTP induction (FIG. 4f), consistent with its modest effect on NMDAR currents in these cells (FIG. 1g).

### Example 4

### Off-State A2aR Antagonism Attenuated LID Induction

In the healthy brain, the strength of iSPN glutamatergic synapses is widely thought to be shaped by the outcome of actions and linked activity in nigrostriatal dopaminergic neurons. The strength of those connections governs the activity of iSPN ensembles and purposeful movement. In the brains of LID mice, where dopaminergic signaling is driven up and then down for hours by non-contingent, pulsatile levodopa administration, this relationship is lost. Results described herein show that this aberrant dopaminergic signaling up-regulates surface GluN2B-NMDARs in iSPNs and enables the induction of plasticity at glutamatergic synapses.

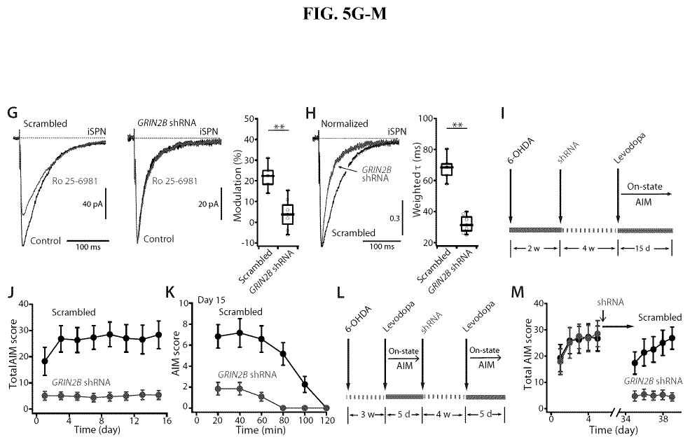

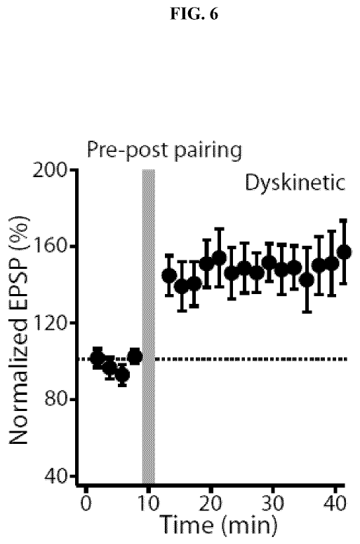

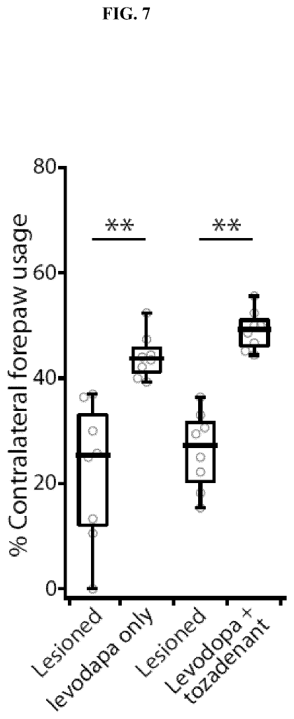

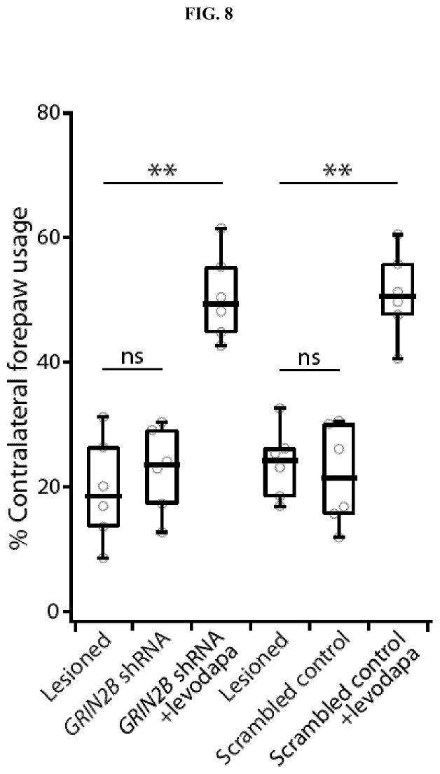

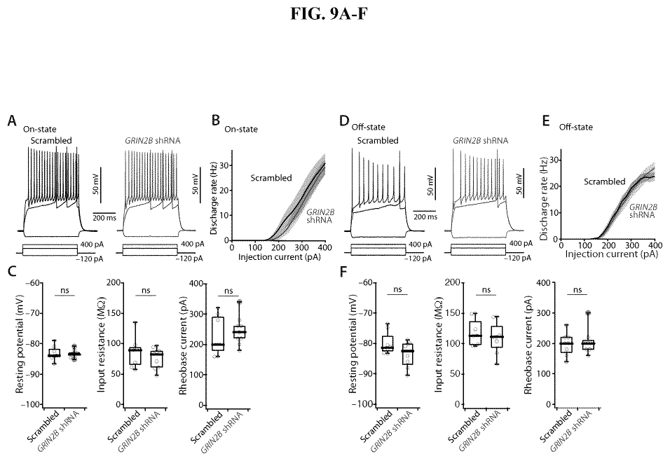

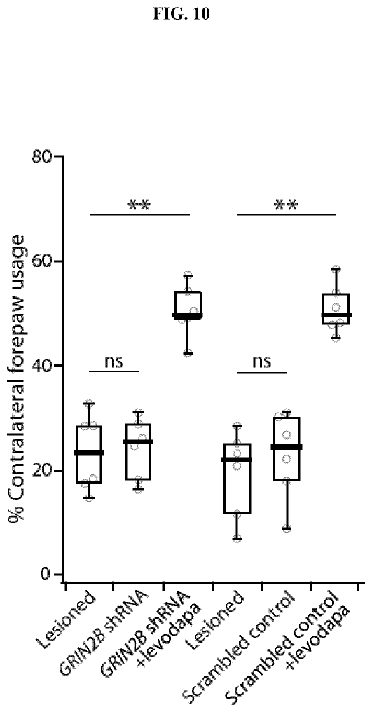

An A2aR antagonist (tozadenant) with good brain bioavailability was given to 6-OHDA lesioned mice during levodopa treatment. If LTP induction in iSPNs was contributing to LID, then antagonizing A2aRs (which prevents LTP induction) should blunt LID induction. An important consideration in these experiments is the pharmacokinetic profile of tozadenant. Previous work has shown that coadministration of levodopa and the A2aR antagonist istradefylline (which has a similar pharmacokinetic profile as tozadenant) was ineffective in ameliorating LID in humans as well as rodent models. However, experiments conducted during development of embodiments herein indicate that the critical time of A2aR signaling in the induction of iSPN synaptic plasticity is in the off-state, after levodopa has cleared, not in the on-state. To test this idea, mice were unilaterally lesioned with 6-OHDA and then randomly assigned to receive treatment with either levodopa (3 mg kg-1, i.p.) and vehicle or levodopa and tozadenant (30 mg kg-1, i.p.). Tozadenant was administered 5-6 hours after levodopa treatment, near the beginning of the off-state (FIG. 5a). AIM scores were recorded following the next dose of levodopa. As predicted, tozadenant significantly attenuated the development of AIMs over the treatment period (FIG. 5b, c). The reduction in dyskinesia scores did not occur at the expense of the anti-akinetic effect of levodopa, as forelimb use asymmetry was equally improved by levodopa when given alone or combined with tozadenant (Supplementary FIG. 2).

### Example 5

### Knocking Down the GluN2B Subunit in iSPNs Blunted Both the Induction and Expression of LID

To test the role of GluN2B up-regulation in iSPNs more directly, a genetic approach was used. A previously validated short hairpin ribonucleic acid (shRNA) sequence targeting GRIN2B mRNA 60 and a scrambled shRNA control were placed in a double-floxed inverse orientation (DIO) element and packaged in an adeno-associated virus (AAV) vector. In unilaterally 6-OHDA lesioned Adora2-Cre mice, the GluN2B-targeted AAV and its control were stereotaxically injected into the DLS (FIG. 5d). About 40% of the neurons in the striatal region injected expressed the FusionRed reporter, consistent with selective expression in iSPNs (FIG. 5e, f). To test for the efficacy of the knockdown, an antibody to GluN2B was used to examine the extent to which expression of the shRNA reduced expression of the protein. As expected, SPNs expressing the FusionRed reporter had little or no detectable GluN2B immunoreactivity (FIG. 5e, f). In addition, electrophysiological recordings found that GRIN2B knockdown in infected iSPNs occluded Ro 25-6981 sensitive current blockade and speeded up their de-activation (FIG. 5g, h). The intrinsic excitability of iSPNs (measured using intrasomatic current injection) was not altered by GluN2B knockdown in the LID off-state (Supplementary FIG. 3). Lastly, knocking down GRIN2B had no effect on the forelimb usage asymmetry produced by 6-OHDA lesioning (Supplementary FIG. 4)

Next, the impact of GRIN2B knockdown on dyskinetic behavior was examined. Four weeks after DLS injection of the AAV GRIN2B shRNA and its control, mice were given levodopa (3 mg kg-1, i.p.) daily for 15 days and LID AIM scores were rated every other day. Knocking down GRIN2B mRNA selectively in iSPNs significantly reduced peak AIM scores induced by levodopa and shortened the duration of dyskinetic behaviors (FIG. 5i-k). The reduction in dyskinesia scores did not occur at the expense of the anti-parkinsonian effects of levodopa, as forelimb use asymmetry was improved by levodopa in mice injected with either GRIN2B shRNA or its scrambled control (lesioned n=6 mice; levodopa+GRIN2B shRNA n=6 mice, p<0.01, Wilcoxon test; lesioned n=6 mice; levodopa+GRIN2B shRNA scrambled n=6 mice; p<0.01, Wilcoxon test) (Supplementary FIG. 4).

Experiments were conducted during development of embodiments herein to assess the ability of iSPN-specific targeting of GluN2B to reduce the expression of established dyskinetic behavior. To answer this question, a cohort of well-lesioned Adora2-Cre mice were given dyskinesiogenic doses of levodopa (3 mg kg-1, i.p.) for 5 days to establish a stable LID. Then, the GRIN2B shRNA and scrambled shRNA vectors were injected into the DLS. Four weeks later, mice were challenged with levodopa. Mice injected with the control shRNA AAV exhibited a robust dyskinesia in response to levodopa after the ‘drug holiday’. However, mice in which GRIN2B had been knocked down exhibited little or no dyskinesia in response to the levodopa challenge (FIG. 5l, m). The reduction in dyskinesia did not compromise the pro-movement effect of levodopa (lesioned n=6 mice; levodopa+GRIN2B shRNA n=6 mice, p<0.01, Wilcoxon test; lesioned n=6 mice; levodopa+GRIN2B shRNA scrambled n=6 mice; p<0.01, Wilcoxon test) (Supplementary FIG. 5). Thus, blunting the contribution of GluN2B subunits to iSPN NMDARs effectively reduced both the induction and expression of LID, without diminishing the benefits of levodopa.

SEQUENCES

SEQ ID NO: 1-GRIN2B shRNA-1
GCTGGTGATAATCCTTCTGAA
SEQ ID NO: 2-GRIN2B shRNA-2
CTTCAGTGAAGATGGCTACCA
SEQ ID NO: 3-GRIN2B
MKPRAECCSPKFWLVLAVLAVSGSRARSQKSPPSIGIAVILVGTSDEVA
IKDAHEKDDFHHLSVVPRVELVAMNETDPKSIITRICDLMSDRKIQGVV
FADDTDQEAIAQILDFISAQTLTPILGIHGGSSMIMADKDESSMFFQFG
PSIEQQASVMLNIMEEYDWYIFSIVTTYFPGYQDFVNKIRSTIENSFVG
WELEEVLLLDMSLDDGDSKIQNQLKKLQSPIILLYCTKEEATYIFEVAN
SVGLTGYGYTWIVPSLVAGDTDTVPAEFPTGLISVSYDEWDYGLPARVR
DGIAIITTAASDMLSEHSFIPEPKSSCYNTHEKRIYQSNMLNRYLINVT
FEGRNLSFSEDGYQMHPKLVIILLNKERKWERVGKWKDKSLQMKYYVWP
RMCPETEEQEDDHLSIVTLEEAPFVIVESVDPLSGTCMRNTVPCQKRIV
TENKTDEEPGYIKKCCKGFCIDILKKISKSVKFTYDLYLVTNGKHGKKI
NGTWNGMIGEVVMKRAYMAVGSLTINEERSEVVDFSVPFIETGISVMVS
RSNGTVSPSAFLEPFSADVWVMMFVMLLIVSAVAVFVFEYFSPVGYNRC
LADGREPGGPSFTIGKAIWLLWGLVENNSVPVQNPKGTTSKIMVSVWAF
FAVIFLASYTANLAAFMIQEEYVDQVSGLSDKKFQRPNDFSPPFRFGTV
PNGSTERNIRNNYAEMHAYMGKFNQRGVDDALLSLKTGKLDAFIYDAAV
LNYMAGRDEGCKLVTIGSGKVFASTGYGIAIQKDSGWKRQVDLAILQLF
GDGEMEELEALWLTGICHNEKNEVMSSQLDIDNMAGVFYMLGAAMALSL
ITFICEHLFYWQFRHCFMGVCSGKPGMVFSISRGIYSCIHGVAIEERQS
VMNSPTATMNNTHSNILRLLRTAKNMANLSGVNGSPQSALDFIRRESSV
YDISEHRRSFTHSDCKSYNNPPCEENLFSDYISEVERTFGNLQLKDSNV
YQDHYHHHHRPHSIGSASSIDGLYDCDNPPFTTQSRSISKKPLDIGLPS
SKHSQLSDLYGKFSFKSDRYSGHDDLIRSDVSDISTHTVTYGNIEGNAA
KRRKQQYKDSLKKRPASAKSRREFDEIELAYRRRPPRSPDHKRYFRDKE
GLRDFYLDQFRTKENSPHWEHVDLTDIYKERSDDFKRDSVSGGGPCTNR
SHIKHGTGDKHGVVSGVPAPWEKNLTNVEWEDRSGGNFCRSCPSKLHNY
STTVTGQNSGRQACIRCEACKKAGNLYDISEDNSLQELDQPAAPVAVTS
NASTTKYPQSPTNSKAQKKNRNKLRRQHSYDTFVDLQKEEAALAPRSVS
LKDKGRFMDGSPYAHMFEMSAGESTFANNKSSVPTAGHHHHNNPGGGYM
LSKSLYPDRVTQNPFIPTFGDDQCLLHGSKSYFFRQPTVAGASKARPDF
RALVTNKPVVSALHGAVPARFQKDICIGNQSNPCVPNNKNPRAFNGSSN
GHVYEKLSSIESDV

### REFERENCES

The following references are herein incorporated by reference in their entireties.
1. Hornykiewicz, O. Dopamine (3-hydroxytyramine) and brain function. Pharmacol Rev 18, 925-964 (1966).
2. Kish, S. J., Shannak, K. & Hornykiewicz, O. Uneven pattern of dopamine loss in the striatum of patients with idiopathic Parkinson's disease. Pathophysiologic and clinical implications. N Engl J Med 318, 876-880 (1988).
3. Yin, H. H. & Knowlton, B. J. The role of the basal ganglia in habit formation. Nat Rev Neurosci 7, 464-476 (2006).
4. Cenci, M. A. & Kumar, A. Cells, pathways, and models in dyskinesia research. Curr Opin Neurobiol 84, 102833 (2024).
5. Jenner, P. Molecular mechanisms of L-DOPA-induced dyskinesia. Nat Rev Neurosci 9, 665-677 (2008).
6. Cenci, M. A., Riggare, S., Pahwa, R., Eidelberg, D. & Hauser, R. A. Dyskinesia matters. Mov Disord 35, 392-396 (2020).
7. Picconi, B., et al. Loss of bidirectional striatal synaptic plasticity in L-DOPA-induced dyskinesia. Nat Neurosci 6, 501-506 (2003).
8. Picconi, B., et al. Inhibition of phosphodiesterases rescues striatal long-term depression and reduces levodopa-induced dyskinesia. Brain 134, 375-387 (2011).
9. Shen, W., et al. M4 muscarinic receptor signaling ameliorates striatal plasticity deficits in models of L-DOPA-Induced dyskinesia. Neuron 88, 762-773 (2015).
10. Reynolds, J. N., Hyland, B. I. & Wickens, J. R. A cellular mechanism of reward-related learning. Nature 413, 67-70 (2001).
11. Shen, W., Flajolet, M., Greengard, P. & Surmeier, D. J. Dichotomous dopaminergic control of striatal synaptic plasticity. Science 321, 848-851 (2008).
12. Yagishita, S., et al. A critical time window for dopamine actions on the structural plasticity of dendritic spines. Science 345, 1616-1620 (2014).
13. Fieblinger, T., et al. Cell type-specific plasticity of striatal projection neurons in parkinsonism and L-DOPA-induced dyskinesia. Nat Commun 5, 5316 (2014).
14. Ryan, M. B., Bair-Marshall, C. & Nelson, A. B. Aberrant Striatal Activity in Parkinsonism and Levodopa-Induced Dyskinesia. Cell Rep 23, 3438-3446 e3435 (2018).
15. Zhang, Y., et al. Aberrant restoration of spines and their synapses in L-DOPA-induced dyskinesia: involvement of corticostriatal but not thalamostriatal synapses. Journal of Neuroscience 33, 11655-11667 (2013).
16. Alcacer, C., et al. Chemogenetic stimulation of striatal projection neurons modulates responses to Parkinson&apos;s disease therapy. J Clin Invest 127, 720-734 (2017).
17. Klaus, A., Alves da Silva, J. & Costa, R. M. What, If, and When to Move: Basal Ganglia Circuits and Self-Paced Action Initiation. Annu Rev Neurosci 42, 459-483 (2019).
18. Lerner, T. N. & Kreitzer, A. C. Neuromodulatory control of striatal plasticity and behavior. Current Opinion in Neurobiology 21, 322-327 (2011).
19. Iino, Y., et al. Dopamine D2 receptors in discrimination learning and spine enlargement. Nature 579, 555-560 (2020).
20. Bourque, M., et al. AV-101, a Pro-Drug Antagonist at the NMDA Receptor Glycine Site, Reduces L-Dopa Induced Dyskinesias in MPTP Monkeys. Cells 11 (2022).
21. Frouni, I. & Huot, P. Glutamate modulation for the treatment of levodopa induced dyskinesia: a brief review of the drugs tested in the clinic. Neurodegener Dis Manag 12, 203-214 (2022).
22. Oh, J. D. & Chase, T. N. Glutamate-mediated striatal dysregulation and the pathogenesis of motor response complications in Parkinson's disease. Amino acids 23, 133-139 (2002).
23. Fino, E., Glowinski, J. & Venance, L. Bidirectional activity-dependent plasticity at corticostriatal synapses. Journal of Neuroscience 25, 11279-11287 (2005).
24. Pawlak, V., Wickens, J. R., Kirkwood, A. & Kerr, J. N. D. Timing is not Everything: Neuromodulation Opens the STDP Gate. Frontiers in Synaptic Neuroscience 2, 146 (2010).
25. Suarez, L. M., et al. L-DOPA treatment selectively restores spine density in dopamine receptor D2-expressing projection neurons in dyskinetic mice. Biol Psychiatry 75, 711-722 (2016).
26. Schallert, T., Fleming, S. M., Leasure, J. L., Tillerson, J. L. & Bland, S. T. CNS plasticity and assessment of forelimb sensorimotor outcome in unilateral rat models of stroke, cortical ablation, parkinsonism and spinal cord injury. Neuropharmacology 39, 777-787 (2000).
27. Shen, W., et al. Striatal Kir2 K+ channel inhibition mediates the ant dyskinetic effects of amantadine. J Clin Invest 130, 2593-2601 (2020).
28. Andreoli, L., Abbaszadeh, M., Cao, λ. & Cenci, M. A. Distinct patterns of dyskinetic and dystonic features following D1 or D2 receptor stimulation in a mouse model of parkinsonism. Neurobiol Dis 157, 105429 (2021).
29. Sheng, M., Cummings, J., Roldan, L. A., Jan, Y. N. & Jan, L. Y. Changing subunit composition of heteromeric NMDA receptors during development of rat cortex. Nature 368, 144-147 (1994).
30. Paoletti, P., Bellone, C. & Zhou, Q. NMDA receptor subunit diversity: impact on receptor properties, synaptic plasticity and disease. Nature Reviews Neuroscience 14, 383-400 (2013).
31. Traynelis, S. F., et al. Glutamate receptor ion channels: structure, regulation, and function. Pharmacol Rev 62, 405-496 (2010).
32. Gray, J. A., et al. Distinct modes of AMPA receptor suppression at developing synapses by GluN2A and GluN2B: single-cell NMDA receptor subunit deletion in vivo. Neuron 71, 1085-1101 (2011).
33. Hansen, K. B., Ogden, K. K., Yuan, H. & Traynelis, S. F. Distinct functional and pharmacological properties of Triheteromeric GluN1/GluN2A/GluN2B NMDA receptors. Neuron 81, 1084-1096 (2014).
34. Rauner, C. & Kohr, G. Triheteromeric NR1/NR2A/NR2B receptors constitute the major Nmethyl-D-aspartate receptor population in adult hippocampal synapses. The Journal of Biological Chemistry 286, 7558-7566 (2011).
35. Sun, W., Hansen, K. B. & Jahr, C. E. Allosteric Interactions between NMDA Receptor Subunits Shape the Developmental Shift in Channel Properties. Neuron 94, 58-64.e53 (2017).
36. Tovar, K. R., McGinley, M. J. & Westbrook, G. L. Triheteromeric NMDA receptors at hippocampal synapses. Journal ofNeuroscience 33, 9150-9160 (2013).
37. Stroebel, D., Casado, M. & Paoletti, P. Triheteromeric NMDA receptors: from structure to synaptic physiology. Curr Opin Physiol 2, 1-12 (2018).
38. Wu, E., Zhang, J., Zhang, J. & Zhu, S. Structural insights into gating mechanism and allosteric regulation of NMDA receptors. Curr Opin Neurobiol 83, 102806 (2023).
39. Lu, W., Du, J., Goehring, A. & Gouaux, E. Cryo-EM structures of the triheteromeric NMDA receptor and its allosteric modulation. Science 355 (2017).
40. Chen, N., et al. Subtype-specific enhancement of NMDA receptor currents by mutant huntingtin. J Neurochem 72, 1890-1898 (1999).
41. Vicini, S., et al. Functional and Pharmacological Differences Between Recombinant NMethyl-d-Aspartate Receptors. Journal of Neurophysiology 79, 555-566 (1998).
42. Cull-Candy, S., Brickley, S. & Farrant, M. NMDA receptor subunits: diversity, development and disease. Curr Opin Neurobiol 11, 327-335 (2001).
43. Fayyazuddin, A., Villarroel, A., Le Goff, A., Lerma, J. & Neyton, J. Four residues of the extracellular N-terminal domain of the NR2A subunit control high-affinity Zn2+ binding to NMDA receptors. Neuron 25, 683-694 (2000).
44. Jahr, C. E. High probability opening of NMDA receptor channels by L-glutamate. Science 255, 470-472 (1992).
45. Hall, B. J., Ripley, B. & Ghosh, A. NR2B signaling regulates the development of synaptic AMPA receptor current. Journal of Neuroscience 27, 13446-13456 (2007).
46. Liao, D., Hessler, N. A. & Malinow, R. Activation of postsynaptically silent synapses during pairing-induced LTP in CA1 region of hippocampal slice. Nature 375, 400-404 (1995).
47. Higley, M. J. & Sabatini, B. L. Competitive regulation of synaptic Ca2+ influx by D2 dopamine and A2A adenosine receptors. Nat Neurosci 13, 958-966 (2010).
48. Pawlak, V. & Kerr, J. N. D. Dopamine receptor activation is required for corticostriatal spike-timing-dependent plasticity. Journal of Neuroscience 28, 2435-2446 (2008).
49. Dias, R. B., Rombo, D. M., Ribeiro, J. A., Henley, J. M. & Sebastiao, A. M. Adenosine: setting the stage for plasticity. Trends Neurosci 36, 248-257 (2013).
50. Yashiro, K. & Philpot, B. D. Regulation of NMDA receptor subunit expression and its implications for LTD, LTP, and metaplasticity. Neuropharmacology 55, 1081-1094 (2008).
51. Calon, F., et al. Increased adenosine A2A receptors in the brain of Parkinson's disease patients with dyskinesias. Brain: a journal of neurology 127, 1075-1084 (2004).
52. Mishina, M., et al. Adenosine A2A receptors measured with [11C]TMSX PET in the striata of Parkinson's disease patients. PLoS ONE 6, e17338 (2011).
53. Ramlackhansingh, A. F., et al. Adenosine 2A receptor availability in dyskinetic and nondyskinetic patients with Parkinson disease. Neurology 76, 1811-1816 (2011).
54. Tomiyama, M., et al. Upregulation of striatal adenosine A2A receptor mRNA in 6-hydroxydopamine-lesioned rats intermittently treated with L-DOPA. Synapse 52, 218 222 (2004).
55. Cui, G., et al. Concurrent activation of striatal direct and indirect pathways during action initiation. Nature 494, 238-242 (2013).
56. Parker, J. G., et al. Diametric neural ensemble dynamics in parkinsonian and dyskinetic states. Nature 557, 177-182 (2018).
57. Jenner, P., Mori, A., Aradi, S. D. & Hauser, R. A. Istradefylline—a first generation adenosine A(2A) antagonist for the treatment of Parkinson's disease. Expert Rev Neurother 21, 317-333 (2021).
58. Lundblad, M., Vaudano, E. & Cenci, M. A. Cellular and behavioural effects of the adenosine A2a receptor antagonist KW-6002 in a rat model of 1-DOPA-induced dyskinesia. Journal of Neurochemistry 84, 1398-1410 (2003).
59. Preston, Z., et al. Adenosine receptor expression and function in rat striatal cholinergic interneurons. Br J Pharmacol 130, 886-890 (2000).
60. Wang, Y. Q., et al. Neuropathic pain generates silent synapses in thalamic projection to anterior cingulate cortex. Pain 162, 1322-1333 (2021).
61. Brotchie, J. M. Nondopaminergic mechanisms in levodopa-induced dyskinesia. Movement Disorders 20, 919-931 (2005).
62. Calon, F., Rajput, A. H., Homykiewicz, O., Bedard, P. J. & Paolo, T. D. Levodopa-induced motor complications are associated with alterations of glutamate receptors in Parkinson's disease. Neurobiology of Disease 14, 404-416 (2003).
63. Chase, T. N. Striatal plasticity and extrapyramidal motor dysfunction. Parkinsonism & Related Disorders 10, 305-313 (2004).
64. Du, K., Asahara, H., Jhala, U.S., Wagner, B. L. & Montminy, M. Characterization of a CREB gain-of-function mutant with constitutive transcriptional activity in vivo. Molecular and Cellular Biology 20, 4320-4327 (2000).
65. Hurley, M. J., Jackson, M. J., Smith, L. A., Rose, S. & Jenner, P. Immunoautoradiographic analysis of NMDA receptor subunits and associated postsynaptic density proteins in the brain of dyskinetic MPTP-treated common marmosets. The European Journal of Neuroscience 21, 3240-3250 (2005).
66. Gardoni, F., et al. Targeting NR2A-containing NMDA receptors reduces L-DOPA-induced dyskinesias. Neurobiology of Aging 33, 2138-2144 (2012).
67. Paille, V., et al. Distinct levels of dopamine denervation differentially alter striatal synaptic plasticity and NMDA receptor subunit composition. Journal of Neuroscience 30, 14182-14193 (2010).
68. Gardoni, F. & Luca, M. D. Targeting glutamatergic synapses in Parkinson's disease. Current Opinion in Pharmacology 20, 24-28 (2015).
69. Calon, F., et al. Alteration of glutamate receptors in the striatum of dyskinetic 1-methyl-4-phenyl-1,2,3,6-tetrahydropyridine-treated monkeys following dopamine agonist treatment. Prog Neuropsychopharmacol Biol Psychiatry 26, 127-138 (2002).
70. Hallett, P. J., et al. Alterations of striatal NMDA receptor subunits associated with the development of dyskinesia in the MPTP-lesioned primate model of Parkinson's disease. Neuropharmacology 48, 503-516 (2005).
71. Picconi, B., et al. Striatal synaptic changes in experimental parkinsonism: role of NMDA receptor trafficking in PSD. Parkinsonism Relat Disord 14 Suppl 2, S145-149 (2008).
72. Lau, C. G. & Zukin, R. S. NMDA receptor trafficking in synaptic plasticity and neuropsychiatric disorders. Nat Rev Neurosci 8, 413-426 (2007).
73. Melchior, J. R., Ferris, M. J., Stuber, G. D., Riddle, D. R. & Jones, S. R. Optogenetic versus electrical stimulation of dopamine terminals in the nucleus accumbens reveals local modulation of presynaptic release. J Neurochem 134, 833-844 (2015).
74. Smith, Y., et al. The thalamostriatal system in normal and diseased states. Front Syst Neurosci 8, 5 (2014).
75. Trepanier, C. H., Jackson, M. F. & MacDonald, J. F. Regulation of NMDA receptors by the tyrosine kinase Fyn. FEBS J 279, 12-19 (2012).
76. Zhang, W. B., et al. Fyn Kinase regulates GluN2B subunit-dominant NMDA receptors in human induced pluripotent stem cell-derived neurons. Sci Rep 6, 23837 (2016).
77. Ba, M., Kong, M. & Ma, G. Postsynaptic density protein 95-regulated NR2B tyrosine phosphorylation and interactions of Fyn with NR2B in levodopa-induced dyskinesia rat models. Drug Des Devel Ther 9, 199-206 (2015).
78. Dunah, A. W., et al. Dopamine D1-dependent trafficking of striatal N-methyl-D-aspartate glutamate receptors requires Fyn protein tyrosine kinase but not DARPP-32. Mol Pharmacol 65, 121-129 (2004).
79. Mao, L. M., Demehri, S. & Wang, J. Q. Upregulation of Src Family Tyrosine Kinases in the Rat Striatum by Adenosine A(2A) Receptors. J Mol Neurosci 72, 802-811 (2022).
80. Schwarzschild, M. A., Agnati, L., Fuxe, K., Chen, J. F. & Morelli, M. Targeting adenosine A2A receptors in Parkinson's disease. Trends Neurosci 29, 647-654 (2006).
81. Sanz-Blasco, S., et al. The Kinase Fyn As a Novel Intermediate in L-DOPA-Induced Dyskinesia in Parkinson's Disease. Mol Neurobiol 55, 5125-5136 (2018).
82. Hanse, E., Seth, H. & Riebe, I. AMPA-silent synapses in brain development and pathology. Nature Reviews Neuroscience 14, 839-850 (2013).
83. Kerchner, G. A. & Nicoll, R. A. Silent synapses and the emergence of a postsynaptic mechanism for LTP. Nature Reviews Neuroscience 9, 813-825 (2008).
84. Chen, W., Wang, H., Wei, H., Gu, S. & Wei, H. Istradefylline, an adenosine A₂A receptor antagonist, for patients with Parkinson's Disease: a meta-analysis. Journal of the Neurological Sciences 324, 21-28 (2013).
85. Kondo, T., Mizuno, Y. & Group, J. I. S. A long-term study of istradefylline safety and efficacy in patients with Parkinson disease. Clinical Neuropharmacology 38, 41-46 (2015).
86. Castela, I., et al. Selective activation of striatal indirect pathway suppresses levodopa induced-dyskinesias. Neurobiol Dis 176, 105930 (2023).
87. Lee, H. K. & Kirkwood, A. Mechanisms of Homeostatic Synaptic Plasticity in vivo. Front Cell Neurosci 13, 520 (2019).
88. Courtney, C. D., Pamukcu, A. & Chan, C. S. Cell and circuit complexity of the external globus pallidus. Nat Neurosci 26, 1147-1159 (2023).
89. Corbit, V. L., et al. Pallidostriatal Projections Promote beta Oscillations in a Dopamine-Depleted Biophysical Network Model. J Neurosci 36, 5556-5571 (2016).
90. Chan, K. Y., et al. Engineered AAVs for efficient noninvasive gene delivery to the central and peripheral nervous systems. Nat Neurosci 20, 1172-1179 (2017).
91. Claringbould, A. & Zaugg, J. B. Enhancers in disease: molecular basis and emerging treatment strategies. Trends Mol Med 27, 1060-1073 (2021).
92. Francardo, V., et al. Impact of the lesion procedure on the profiles of motor impairment and molecular responsiveness to L-DOPA in the 6-hydroxydopamine mouse model of Parkinson's disease. Neurobiology of Disease 42, 327-340 (2011).
93. Cenci, M. A. & Lundblad, M. Ratings of L-DOPA-induced dyskinesia in the unilateral 6-OHDA lesion model of Parkinson's disease in rats and mice. Current Protocols in Neuroscience Chapter 9, Unit 9.25 (2007).
94. Zhai, S., Ark, E. D., Parra-Bueno, P. & Yasuda, R. Long-distance integration of nuclear ERK signaling triggered by activation of a few dendritic spines. Science 342, 1107-1111 (2013).
95. Fellmann, C., et al. An optimized microRNA backbone for effective single-copy RNAi. Cell Rep 5, 1704-1713 (2013).
96. Yu, X., et al. Wakefulness Is Governed by GABA and Histamine Cotransmission. Neuron 87, 164-178 (2015).

What is claimed is:

1. A method of treating Levodopa-induced dyskinesia (LID), in a subject in need thereof, the method comprising: administering a GRIN2B inhibitor to the subject.

2. The method of claim 1, wherein the GRIN2B inhibitor is administered into an indirect pathway spiny projection neuron (iSPN).

3. The method of claim 1, wherein the GRIN2B inhibitor reduces GluN2B protein levels.

4. The method of claim 1, wherein the GRIN2B inhibitor is an inhibitor of GRIN2B expression.

5. The method of claim 4, wherein the GRIN2B inhibitor is a nucleic acid inhibitor of GRIN2B expression.

6. The method of claim 1, wherein the nucleic acid GRIN2B inhibitor is a microRNA (miRNA), a small interfering (siRNA), a short hairpin RNA (shRNA), an anti-sense RNA (asRNA), a competing endogenous RNA (ceRNA), a long non-coding RNA (lncRNA) and a ribozyme.

7. The method of claim 6, wherein the shRNA has at least 70% sequence identity to SEQ ID NO: 1 or 2.

8. The method of claim 7, wherein the nucleic acid GRIN2B inhibitor is a shRNA having at 100% sequence identity to SEQ ID NO: 1 or 2.

9. The method of claim 5, wherein the nucleic acid GRIN2B inhibitor is within a vector.

10. The method of claim 9, wherein the vector is a viral vector.

11. The method of claim 10, wherein the viral vector is an adeno-associated viral vector (AAV), a adenoviral vector, a lentiviral vector, a non-viral vector, a human-compatible vector, a herpes simplex virus vector and a retroviral vector.

12. The method of claim 1, wherein the subject is afflicted with the neurodegenerative disease associated with an upregulation of GluN2B-containing N-methyl-D-aspartate receptors (NMDARs).

13. The method of claim 12, wherein the neurodegenerative disease is Parkinson's disease.

14. The method of claim 1, wherein the subject is a human.

15. The method of claim 1, wherein the subject was administered or is currently being a therapeutic agent.

16. The method of claim 15, wherein the therapeutic agent administered is levodopa (L-3,4-dihydroxyphenylalanine).

17. The method of claim 1, wherein the GRIN2B inhibitor is administered by an intraperitoneal (IP) injection.

18. The method of claim 1, wherein said GRIN2B inhibitor is administered repeatedly to the subject.

19. The method of claim 1, wherein said GRIN2B inhibitor is administered during the low levels of DA to the subject after levodopa is administered.

20. A method for treating PD comprising co-administering levodopa and an inhibitor of GRIN2B.


SEQUENCE LISTING (3 sequences)

SEQ ID NO: 1 - RNA, 21 nt, Synthetic construct
  source 1..21: mol_type = other RNA
gctggtgataatccttctgaa

SEQ ID NO: 2 - RNA, 21 nt, Synthetic construct
  source 1..21: mol_type = other RNA
cttcagtgaagatggctacca

SEQ ID NO: 3 - protein, 1484 aa, Homo sapiens [sequence omitted]